(19) United States
(12) Patent Application Publication
(10) Pub. No.: US 2007/0077547 A1
(43) Pub. Date: Apr. 5, 2007

(54) Assay assembly

(75) Inventors: Igor Shvets, Dublin (IE); Dmitri Kashanin, Dublin (IE); Dermot Kelleher, Dublin (IE); Vivienne Williams, Wexford (IE)

Correspondence Address: BIRCH STEWART KOLASCH & BIRCH, PO BOX 747, FALLS CHURCH, VA, 22040-0747, US

(73) Assignee: THE PROVOST FELLOWS AND SCHOLARS OF THE COLLEGE OF THE HOLY AND UNDIVIDED TRINITY OF QUEEN ELIZABETH, DUBLIN 2 (IE)

(21) Appl. No.: 10/500,277

(22) PCT Filed: Jul. 26, 2002
(86) PCT No.: PCT/IE02/00107

§ 371 Date: Feb. 2, 2005

(30) Foreign Application Priority Data
Dec. 31, 2001 (EP) 02017036.1

(51) Int. Cl. C12M 1/34; C12Q 1/00

(52) U.S. Cl. 435/4; 435/287.2; 257/E51.02

(57) ABSTRACT

A cell based assay assembly includes a biochip assembly, a liquid delivery unit and detection and recording equipment is used for conducting an assay on a biological cell as it is delivered through the biochip assembly. The biochip assembly includes a plurality of separate biochips each comprising a microchannel with input and output ports, separate reservoir wells are provided on the biochip assembly and are periodically connected to the liquid delivery unit by removable separate disclosed transfer conduits. The input and output ports of the biochip are also periodically connected to the delivery unit by the conduit. The liquid delivery unit includes a liquid link assembly and a positive displacement pump such as a syringe pump. The liquid link assembly includes pressure compressible means which acts to smooth out pressure rises by initially contacting and then expanding to in turn dispense a steady liquid delivery output below 10μ per minute.

### INTRODUCTION

The present invention relates to a biochip assembly for a cell based assay of the type comprising a biochip having an elongate microchannel, an inlet port mounted adjacent a proximal end of the microchannel and an outlet port mounted adjacent a distal end of the microchannel and a liquid delivery unit for the transmission of liquid through the biochip, the liquid delivery unit having at least one liquid delivery port. Further, the invention comprises a cell based assay assembly incorporating the biochip assembly. Finally, there is provided a method of conducting a biological cell based assay on a cell based assembly.

Biological assays are performed every day in laboratories. Assays involving cells, e.g. cell suspensions are becoming increasingly important. One of the reasons of increasing emphasis placed on the cell-based assays is in appreciation of the fact that functions of many biological molecules, e.g. proteins can only be studied when the molecules are placed in their natural environment, i.e. the cell. While a considerable amount of attention has naturally been placed on such biological cell assaying for humans, this is also becoming more important in the field of animal welfare and plant production.

Generally the aim of the cell-based assay is to establish response of cells to a biochemical experiment. Preferably the experiment should mimic the in-vivo situation as closely as possible to make the experiment more meaningful and credible. In most cases it is desirable to perform a number of biological experiments simultaneously in parallel in order to increase productivity. For example, these could be assays with several cell lines in parallel whereby each cell line is involved in the same kind of biochemical experiment e.g. in a separate well. Alternatively, these could be assays involving the same cell line in several different biological experiments, for example, the same cell line tested against several drug candidates in parallel or against several concentrations of the same drug candidate. Typically the cell-based assays are currently performed in well plates. For example in a 96 well plate each well can contain a separate experiment involving cells. As will be explained in detail further this kind of environment is far from the natural environment for a cell meaning that e.g. results of many experiments may misrepresent the natural response of the cell to a particular drug candidate.

Below we describe a number of assays related to cell motility, migration and binding where it is vital to perform the assays in the regime of continuous flow.

A rapidly advancing research area in biology is the study of cell receptor-ligand interactions resulting in cell-substratum and cell-cell adhesion followed by subsequent cell migration. The pre-requisite to transendothelial migration of certain cell lines into sites of infection is paramount to the study of inflammatory diseases. This can be briefly summarised as cell flow and rolling, tethering and activation of integrin receptors which is a key recognition step, attachment to the endothelial ligands via activated integrins and finally transendothelial migration or diapedesis. Unfortunately, to date, most of the assay techniques are not particularly successful for the study of these mechanisms. Currently, the majority of studies involving cell rolling and chemokine induced cellular arrest have utilised capillary systems wherein cell flow and shear stress are controlled utilising syringe pumps. Such observations are constrained by a number of factors. Firstly, the relative large (>100 μm) size of the standard glass capillaries limits the physiological analogies to the proximal microvascular regions. Secondly, such studies can only be utilised to study single end-points and cannot be utilised to examine cell choices in migration. Thirdly, optical aberrations related to the spherical geometry of the glass capillary sections limit stage-related in situ (post-fixation) analysis of the intracellular structures (cytoskeleton and signalling molecules). Finally and most importantly, the usual observation periods lie between 5-30 minutes for rolling experiments. Longer studies are required to study subsequent crawling steps on endothelial and extracellular matrix ligands. In this regard, studies relating to the effects of chemokines have largely been limited to cellular arrest on adhesion receptor ligands and have not been extended to the study of cell crawling. For example, specific chemokines have been shown to induce rolling arrest with enhanced binding of lymphocytes to ICAM-1, otherwise known as CD54.

Presently accepted techniques for cell adhesion or binding assays involve the initial coating of a surface of a device with a substrate, typically a protein. Cells are deposited onto the substrate and allowed to settle. Following the settling of the cells, the device is heated to 37° C. and is visually analysed using an inverted microscope, or alternatively it is subjected to a stand-alone heating stage and progression of cell binding can be checked at intervals with the inverted microscope. The duration of these assay experiments may be varied depending on the cell line and choice of substratum. Following cell adhesion, free cells may be washed away and a subsequent cell count may be carried out.

Although these methods provide semi-quantitative information regarding a cell type's affinity for a particular substratum, there is no simple method for quantitative characterisation of binding or methods enabling a prolonged study of cell rolling, the ensuing capture by the substratum and subsequent attachment. Furthermore, direct studies of changes in cell morphology, cell growth and biochemical changes cannot be provided easily with these techniques since, determining the kinetics of attachment and resulting morphological changes requires multiple replicated experiments being analysed at different times.

U.S. Pat. No. 5,998,160 (Berens et al) describes a static assay which, unfortunately, does not have any consideration of cell flow and rolling.

The ability of T-cells circulating in the bloodstream to adhere to the endothelium, switch to a motile phenotype and penetrate through the endothelial layer is recognised as a necessary requirement for the effective in vivo movement or as it is sometimes referred to, trafficking of specific lymphocyte sub-populations. Motility assays are done in combination with attachment assays since following adhesion; cells are expected to switch to the motile phenotype. Motility assays are assessed by estimating the ratio of cells undergoing cytoskeletal rearrangements and the formation of uropods (extension of the trailing tail). One of the major disadvantages of this and the previous adhesion assays is the geometrical design (microscope slides and multiple well chambers), which does not at all resemble the in vivo situation.

The most commonly used cell transmigration assay is a modified “Boyden chamber” assay such as described in U.S. Pat. No. 5,578,492 (Fedun et al). This involves assessing the crossing of a quantity of cells through a microporous membrane under the influence of a chemoattractant, recombinant or cell-derived. Here the diameter of the micropores are less than the diameter of the cells under investigation, such that the cells must deform themselves in order to squeeze through the pores thereby constructing an analogy to the transendothelial migration of cells in physiological circumstances. Once cells are deposited onto the membrane, the chamber can be incubated for intervals over time at a suitable temperature, usually 37°. Following this, the bottom chamber or opposite side of the top chamber may be analysed for cells that have squeezed through the microporous membrane.

U.S. Pat. No. 4,912,057 (Guirguis et al), U.S. Pat. No. 5,284,753 (Goodwin et al), U.S. Pat. No. 5,302,515 (Goodwin et al), U.S. Pat. No. 5,514,555 (Springer et al) and U.S. Pat. No. 5,601,997 (Tchao) are typical examples of these assays. It is suggested that one of the disadvantages of the assays described in those specifications is that the biological process of transmigration through the micropores is difficult to observe due to the geometrical configuration of the apparatus involved. The lens of the optically inverted microscope must be able to focus through the lower chamber and the microporous membrane. This obviously leads to difficulties due to optical aberrations. In effect, the study of the cells morphology changes while transmigrating across the membrane and their subsequent cytoskeletal changes reverting to their former state is a process which is difficult to monitor and record due to limitations with current techniques. in addition, although it is possible to after experiment parameters following the initiation of the experiment, such as the introduction of a second. chemoattractant, recombinant or cell-derived, at some specified time after commencing the experiment, it is not possible to distinguish separate effects from each said chemoattractant.

These assays can be performed for cell biology studies and also in the pharmaceutical industry. The pharmaceutical industry has major problems in the drug screening process and while high throughput screening (HTS) has been extremely successful in the elimination of the large majority of unsuitable drug candidates, it has not progressed significantly beyorid that and usually, after a successful HTS assay, a pharmaceutical company may still have some 10,000 possible drug candidates requiring assessment. This requires animal trials and anything that can be done to reduce the amount of animal trials is to be desired. Thus, there is a need for new techniques for drug testing in the pharmaceutical industry. The current proposals are to screen the physiological response of cells to biologically active compounds such as described in U.S. Pat. No. 6,103,479 (Taylor). This again is a static test. Since the cells are spatially confined with the drug, there may be a reaction but it may not necessarily take place when the cells are free to flow relative to the drug as in, for example, the microcapillaries of the body. There are other disadvantages such as the transport and subsequent reaction of the drug following its injection into the animal. Probably the most important disadvantage is that it does not in any way test in a real situation, drug efficacy. It is important to appreciate that the requirement of the continuous flow is not only relevant for the experiments involving cell motility, binding and migration. There numerous other assays in which the reliability of the data obtained can be greatly improved if the assays are performed under conditions of continuous flow mimicking the in-vivo situation. For example these could be cell toxicity assays, assays involving interaction of cells with biological liquids, assays involving cell-cell interaction and signalling and others.

Our investigations to date have not revealed any techniques for performing assays to test the interaction of a large number of chosen compounds with living cells while the cells or compounds mimic the in vivo situation of continuous flow. Parallel flow chamber allows performing cell-based experiments in the continuous flow regime. The disadvantage of the parallel flow chamber is that it requires significant volumes of the sample for the tests, typically in the range of 100-400 microlitres with dead volumes in the order of millilitres. In many cases using large sample volume is prohibitive. The size of the parallel flow chamber is also too large to allow performing a number of experiments in parallel and in a typical configuration only one experiment is performed at a time. As a result parallel flow chambers failed to become one of key tools in a pharmaceutical company unlike the instruments supporting the high throughput screening applications. Arguably, the most fundamental reason why the cell based assays are currently not performed in the biochip format under the conditions of continuous flow is that there is no adequate pump that can deliver the low flow rates required. liquid flow in a typical parallel flow chamber is maintained using a syringe pump meaning that the typical flow rate in the range of 10-100 microlitres/min is achieved. Therefore, the flow rate of a syringe pump is far too large for the control of the biochip. The system needs to be primed with the volume of sample liquid in the range of several microlitres. Electroosmotics pump can deliver low flow rates in the range of 100 pl/min to 100 nl/min. However, although the electroosmotic pump can handle homogeneous liquids such as DNA solutions, it is not effective for maintaining the flow of cell suspensions. The cell suspensions when pumped with electroosmotic pump tend to block the channels and the reproducible pumping rate is difficult to achieve.

There is a further disadvantage in performing tests using parallel flow chamber. The ratio of active surface to volume in the chamber determined by the diameter of the chamber's channel is much smaller than in a blood capillary. Therefore, as the biochemical processes are determined by this ratio, the result of the experiment in the parallel flow chamber may misrepresent the result of the in-vivo test.

Miniaturization requires new technologies for compound handling, assay development and automation. Drug discovery has consequently been effected by technologies arising from the combination of biotechnology, material sciences and micro-/Nanotechnology. Advances in microfabrication have driven the development of microfluidics. Integration of several miniaturised features on a single chip allow for biological analyses through electrophoresis, fluorescence, immunological detection or electrophysiologically. Through the reduction in size, a corresponding increase in the throughput of handling, processing and analysing of the sample is achieved.

Application of microscalle assays can potentially offer a number of advantages over standard-scale laboratories:
The reduction in required sample and assay down to a few microlitres or even several hundred nanolitres per test.
The faster, and possibly more accurate reaction in micro scale.
The capacity to perform massive parallel analyses.
The possible integration of various laboratory functions like purification, sorting, immobilisation and detection into one chip, ultimately leading to lab-on-a-chip solutions.

Although the miniaturisation of the assay devices gives many advantages, the delivery of low volume samples into the biochip still remains a problem. Particularly, the method of the transferring a plurality of the sample liquids in parallel using only one channel pumping system has not been investigated before. In any system used heretofore such as a parallel flow chambers and DNA biochips samples are prepared outside of the microfluidic structure and transferred onto the biochip subsequently.

For single channel sample handling, essentially, there are two approaches to the preparation and injection of the sample liquids into the biochip. One of them is to deliver the sample through an input port coupler, which usually connects the syringe pump and the parallel flow chamber. In this case the cell suspension or another liquid sample is pumped through the whole pumping system and therefore a sample volume not less then priming volume (dead volume of the pump) has to be used. This sometimes is in the range of hundreds of microlitres. Different sample liquids can also be injected into flow chamber subsequently. Such a handling of the sample is unsuitable for the biochip implementation, when significant sample volume reduction is required.

Simply scaling down of the parallel flow chamber also brings additional difficulties. In this case the fluidic pumping system, usually a conventional syringe pump, which is essentially macroscopic by comparison to the microfluidic structure is connected to the microscopic microchannel. Therefore these two parts need to be correctly matched to avoid the accumulation of the sample at the place of their junction or at the input port and appearance of the air bubbles. The relatively large sample volume required to operate the syringe pump hinders further miniaturisation of the parallel flow chamber.

Another approach to the sample handling used in the conventional DNA biochips operated with electro osmotic pumps, is to integrate sample reservoir wells onto the biochip and directly connect them to the microchannels of the biochip. The sample is stored in these wells all the time during the assay experiment and so called “soaked” to the microchannels. A plurality of wells can be used to deliver several samples into the microchannels of the biochip. One disadvantage of this method is that the one sample liquid cannot be easily replaced in the microwell reservoir without contamination with a previously used sample. To avoid that, the biochip design requires washing procedures. Another disadvantage is that during the experiment, which may run up to several hours, e.g. cell culturing, is that such a low volume of the sample liquid may evaporate from an open microwell reservoir.

It is suggested that the parallel assay analysis system, as used to date, requires a new approach for handling and preparation of the sample liquids. There is a need to simplify the handling of low volume samples in parallel. Also it would be an advantage to be able easily store the sample liquids after the analysis, to be able for example to perform post analysis tests on the cell suspensions treated during the assay experiment The parallel sample delivery and storage system has to be simple in handling and operation.

The miniaturisation process itself leads to a demand for new instruments and tools which can handle biological fluids and reagents with volumes of only a few microlitres.

The issue of sample volume reduction per experiment may seem trivial but it is the decrease in volumes, which ultimately leads to a reduction in costs. With the introduction of the microtechnologies, small molecules such as chemokines, peptides or non-peptide organic compounds that previously would be prohibitively expensive to study can be studied more cheaply than ever.

Numerous developments show a future employment of microfluidics as the platform of choice for drug development and routine clinical diagnostics. Moreover, sorting, separation and analysis of single cells will be essential features of microfluidics. Integration of functions like transport, immobilisation and detection will allow for cell arrays, monitoring whole-cell events online.

Introduction of microchip format for cell-based assays presents a significant demand on High Throughput Screening (HTS) systems. Several issues need to addressed and solved during transition of the assay to a microchip format:
Accurate liquid handling
Minimising evaporation effects
Ensuring comparable assay sensitivity
Tackling the enhanced surface-to-volume ratio
Reproducing the conditions to be encountered on the HTS system as closely as possible.

In drug discovery and HTS, cell-based microarrays are anticipated to herald the post-genomic era, beyond genomics and proteomics. Unlike DNA and protein microarrays, cell microarrays do not require time-consuming purification steps. Moreover, precise cellular positioning will allow for studies of subcellular organisation and microdomain measurements in the intact cell.

Microdevices of the cell array device kind will permit examining enzymatic activity in response to the application of drug candidate compounds. For all mentioned considerations, single cell location and positioning as well as the precise handling of liquids employed in the assay will be key factors in the development of HTS microchips.

It is suggested that the above analysis shows that there is a current requirement for new technologies based on microfluidic chip format capable of performing cell-based assays in parallel with small sample volume down to few microlitres and smaller. There is an additional requirement to perform the assays under the conditions of continuous flow mimicking the in-vivo situation. There is further requirement to provide means for integration of the microfluidic chip with existing technologies for the sample transfer and sample preparation and also to provide means for collection of the cell samples after the continuous flow experiments for the post assay tests.

In these wide areas of application of microstructures, pumping systems play a significant role. Delivering required solutions to the sites of reaction, mixing different liquids, creating gradients of concentration of the reagents, controlling the positions of biological samples, transporting and manipulating them are all tasks, which require a highly accurate pumping system. Despite a major effort in developing pumping systems for a microchannel structure, the problem still remains. Many conventionally used pumping systems are operating with significantly bigger volumes of liquids, therefore they cannot provide pumping accuracy or in some cases adequate pumping speed when it comes to establishing flows inside the microstructures with a microchannel diameter from 5 to 100 μm.

Various constructions of positive displacement pumps, including syringe pumps, positive pressure infusion pumps and peristaltic pumps have been used with capillaries. These are, for example, described in U.S. Pat. No. 4,715,786 (Wolff et al). Syringe pumps with microflow rate capabilities to provide precise and reproducible volumetric flow ranges of the order of 0.1 μl to 1 ml/min. have been described, for example, in U.S. Pat. No. 5,630,706 (Yang) and U.S. Pat. No. 5,656,034 (Kochersperger et al). One of the main objects of these inventions has been to deliver pulse free flow, the problem being that the pressure of the fluid inside the syringe pump changes during the stroke of the syringe pump, which stroke is usually controlled by a stepper motor. Unfortunately, such an operation results in a large pressure surge which alters the volumetric flow rate. For example, Japanese Patent Specification No. 4058074A (Nagataka et al) describes a method to reduce fluctuations of the flow in a syringe pump to provide a more stable flow rate by setting the syringe vertically and forming a gas layer between the front surface of the piston forming the syringe pump and the liquid being pumped. This invention, however, is directed towards relatively large flow rates of the order of microlitres per minute and would be useful for drug infusion but would not be particularly suitable for microchannel structures and the like, where the flow rates are, as mentioned already, substantially less.

U.S. Pat. No. 4,137,913 (Georgi) describes a method of controlling the flow rate by changing the stroke periods. U.S. Pat. No. 5,242,408 (Jhuboo et al) describes a method of controlling pressure inside a syringe pump by measuring the force acting on the plunger and detecting an occlusion. Unfortunately, heretofore, such syringe and positive displacement pumps have been relatively inefficient at delivering fluid flow at rates of the order of nanolitres per minute, which flow rate is required to transport liquids in microchannel structures. Generally, the limitation on the flow rate is the movement accuracy of the various mechanical parts of the syringe pump such as the stepper motor, plunger, valves, and so on. However, syringe pumps used in high pressure liquid chromatography (HPLC) have achieved volumetric flow rates as low as 0.1 μl/min. A typical example of this is described in U.S. Pat. No. 5,630,706 (Yang). However, for commercially available syringe pumps, the linear displacement of the piston or plunger would be several micrometers per step of the motor controlling the pump. Thus, general sealing surface wear makes it impossible to achieve accuracy for shorter displacements.

A further disadvantage of the syringe pump when used for pumping liquids in microchannel structures, is that it cannot deliver a sufficiently low pumping speed for many applications of the structures.

Typically, a syringe pump dispenses 0.6 μl/min for one step of the motor which then has to be delivered into a microchannel structure possibly having a cross sectional diameter of the order of 40 μm which translates into 1.9 mm/sec through the microchannel structure which is much too fast for the observation of biological specimens, detection of proteins, single cells and the creation of low gradients of reagents, which is required in many microfluidic applications. Indeed, one can readily appreciate that at this speed, visual observation is difficult and further-would not allow for the manipulation or sensing of biological samples. Thus, heretofore, positive displacements pumps and in particular, syringe pumps, while very attractive for their simplicity, have not as of yet been useful for these applications. By analysing the state of the art literature, one can conclude that gas/air bubbles are considered generally detrimental to pump performance. As they are compressible, the accuracy of the volume dispensing is compromised by the presence of such bubbles. Therefore, care is usually taken to avoid formation of the bubbles in the system.

Electrokinetic pumps have been proposed for such pumping operations. Pumps based on electroosmotic phenomena are described in U.S. Pat. No. 3,923,426 (Theeuwes et al) and U.S. Pat. No. 5,779,868 (Wallace Parce et al). When a buffer is placed inside a capillary, the inner surface of the capillary acquires a charge. This is due to the ionisation of the wall or adsorption of ions from the buffer. In the case of silicate glass, the surface silanol groups (Si—OH) are ionised to silanoate groups (Si—O). These negatively charged groups attract positively charged cations from the buffer, which form an inner layer of cations at the capillary wall. These cations are not in sufficient density to neutralize all the negative charges, therefore a second layer of cations forms. The inner layer of cations, strongly held by the silanoate groups, forms a fixed layer. The second layer of cations is less strongly held because it is further away from the negative charges, threfore it forms a mobile layer. When an electric field is applied, the mobile layer is pulled toward the cathode. Since ions are in solution, they drag the whole buffer solution with them and cause electroosmotic flow. The distribution of charges due to the formation of charged layers create a potential termed the zeta potential.

This method, originally used for capillary electrophoresis, is recently being used for fluid transport in microstructures and for high speed chromatography in microfluidic chips. However, it still has a number of disadvantages.

The distribution of charges and formation of layers depends on the initial charge of the inner surface of the capillary, which is different for various materials and solutions used. Moreover, it can be reliant on the pH history of the capillary. This makes the control of the zeta potential and therefore electroosmotic flow control a complicated task. The prior art evidences a number of ways to treat the capillary in order to achieve a reproducible flow rate. They indicate that coating the microcapillary with a monomolecular layer of non-cross-linked polyacrylamide can derivatize inner surfaces of a capillary. This coating enhances the osmotic effect and suppresses adsorption of solutes on the walls of the capillary. Others have taught that altering the buffer pH the concentration of the buffer, the addition of surface-active components, such as surfactants, glycerol, etc. or adding various organic modifiers to the buffer solution may alter electroosmotic flow. In some cases this alteration can cause a reverse of electroosmotic flow or its complete cancellation.

Transport of particles in electroosmotic pumping systems is also difficult, due to the fact that during transport they can acquire an electrical charge and can be moved by the electric field, which in some cases causes the flow to reverse.

According to the theory, the mobile layer drags the fluid. As a result electroosmotic flow has a relatively flat flow profile i.e. the flow velocity is fairly uniform across the capillary. When a static pressure is opposed to the electroosmotic flow, the resulting flow can produce a turbulence, which doesn't allow controllable mixing of fluids and biological samples and decreases the speed of electroosmotic flow.

For example, U.S. Pat. No. 4,908,112 (Pace et al) suggests the use of electro-osmotic pumps to move fluids through channels less than 100 microns in diameter. A plurality of electrodes was incorporated in the channels, which were etched into a silicon wafer. An electric field of about 250 volts/cm was required to move the fluid to be tested along the channel. However, when the channel is long, a large voltage needs to be applied to it, which may be impractical for highly integrated structures. This U.S. patent specification suggests that the electrodes be staggered to overcome this problem, so that only small voltages could be applied to a plurality of electrodes. However, this requires careful placement and alignment of a plurality of electrodes along the channel.

Electrohydrodynamic (EHD) pumping of fluids is also known and may be applied to small capillary channels. The principle of pumping here is different from electroosmosis. When a voltage is applied, electrodes in contact with the fluid transfer charge to or from the fluid, such that fluid flow occurs in the direction from the charging electrode to the oppositely charged electrode. Electrohydrodynamic (EHD) pumps can be used for pumping resistive fluids such as organic solvents. U.S. Pat. No. 5,632,876 (Zanzucchi Peter John et al) describes the use of both electroosmotic and electrohydrodynamic fluid movement method to establish flow in microcapillaries for polar and non-polar fluids.

One of common problems that is usually encountered in these two types of liquid pumping system is the appearance of gas bubbles, which are easily obtained during pumping as a result of electrolysis. They normally interfere with particle transport, blocking microstructures, thus requiring additional pressure difference to transport them. Pumping of liquids by pumps based on electroosmosis and electrohydrodynamic phenomena relies on the electrical contact throughout the liquid, which disappears in the presence of bubbles rendering pumping by these methods difficult. Therefore, appearance of gas bubbles inside microfluidic structures poses major problems for such pumps.

Another method of fluid transport in a microfluidic structure is by mechanical micropumps and valves incorporated within the structure such as described in U.S. Pat. No. 5,224,843 (Van Lintel), U.S. Pat. No. 5,759,014 (Van Lintel) and U.S. Pat. No. 5,171,132 (Miyazaki et al).

As described in U.S. Pat. No. 5,759,014 (Van Lintel), the operation of these pumps is greatly influenced by the compressibility of the fluid and the presence of an air bubble inside the pumping chamber. The pumping speed decreases in the presence of a significant air bubble, sometimes even reducing to zero. Procedures for priming these pumps is complicated and requires a vacuum pump or special injection devices, to prevent appearance of bubbles in the micropumps main pumping chamber. Therefore, it is also impractical to use micropumps as a part of disposable microfluidic biochips.

Another method of pumping fluids in microchannel systems is based on centrifugal force caused by rotation of the microchannel structures at speed. In a most common embodiment, the microchannel structure is a disk in a format similar to that of a CD platform. The fluid in this case flows from the centre of rotation to the periphery. Due to opposing surface tension and centrifugal forces at the interface between the fluid medium and air, it is possible to implement valves and switches whose operation is controlled by the angular speed of rotation of the disk. Therefore this method provides a way to facilitate sequential reactions on a chip platform. In US. Pat. No. 6,063,589 (Kellogg Gregory et al), the microsystem platforms are described as having microfluidic components, resistive heating elements, temperature sensing elements, mixing structures and capillarity driven stop valves.

Further, there are described methods for using these microsystem platforms for performing biological, enzymatic, immunological and chemical assays. A rotor with a slip ring capable of transferring electrical signals to and from the microsystem platforms is also described in the specification.

While such centrifugal pumps can provide required flow rates in microfluidic systems and integrate components on a single platform, this method has a number of shortcomings. The fluids can only be transported in one direction and no reversed flow is possible. Control of the flow rate in the individual channels is not possible dynamically, but only by designing a specific geometry of the microchannel structures. Therefore mixing is only possible with predefined ratios. Replacement of one of the fluids for a fluid with a different viscosity requires a change in the design of the structure. For the complicated interconnected channel geometries during the filling process air bubbles may appear in some places. This would require an additional increase in the rotation to pump them and therefore would lead to non-reliable experiments particularly in the case of sequentially executed experiments. This is contrary to the use of pressure pumps where multiple pumps can facilitate a filling process individually for each channel, if required. When a microfluidic structure is rotated at a high speed it becomes impossible to visually observe biological samples, which is very important for a number of applications, for example for the study of cellular responses.

Despite several types of pump methods proposed for pumping liquids in the microchannel structures, there is no simple solution, which can be used in many of the applications utilizing microchannel structures. All methods have some disadvantages, which are more or less significant for different applications. For example, it's not practical to use micromachined pumps in applications of disposable biochips. Integration micromachined pumps with a disposable device would increase the cost of it. In the same example electroosmotic pumps cannot provide a great degree of reliability. It seems to be impractical when every disposable chip needs to be treated before an experiment in order to successfully control electroosmotic velocity.

It will be appreciated that almost with every type of pump, great care is taken to avoid trapping air inside the pump or microfluidic structure, as well as formation of such air/gas bubbles during the experiment. Such bubbles are commonly detrimental to pump's performance.

The present invention is directed towards providing such methods and apparatus for performing such assays. Further, the present invention is also directed towards providing a pumping system and method for pumping liquids in microchannel structures to enable an accurate control of flow for flow rates ranging from 100 picolitres per minute to 10 microlitres per minute. Thus, such a pumping system should be suitable for delivering liquids with biological samples, cells, etc.

While in the description herein, the examples all refer to animal cells and indeed mainly human cells, the invention equally applies to plant cells. The term “sample liquid” refers to a suspension of living cells within a suitable carrier liquid which is effectively a culture medium. More than one cell type may be in suspension. Further, the term “reagent liquid” could be any liquid from a drug under assessment, a poison, a cell nutrient, chemoattractant, a liquid containing other cells in suspension or indeed any liquid who's effect the sample liquid requires assessment.

### STATEMENTS OF THE INVENTION

According to the invention, there is provided a biochip assembly for a cell based assay of the type comprising a biochip (20) having an elongate microchannel, an inlet port mounted adjacent a proximal end of the microchannel and an outlet port mounted adjacent a distal end of the microchannel and a liquid delivery unit for the transmission of liquid through the biochip, the liquid delivery unit having at least one liquid delivery port characterised in that there is provided:
a plurality of separate biochips;
at least one separate reservoir well for each biochip which is not permanently fluidically coupled thereto; and
a plurality of removable separate enclosed transfer conduits for releasable connection of some of the ports and some of the ports and wells.

In one embodiment of the invention, the liquid delivery unit has a separate delivery port for each biochip. One or more wells are provided for each biochip.

In one embodiment of the invention, there are two sets of at least two wells, one set adjacent the inlet port and the other set adjacent the outlet port.

In another embodiment of the invention, the transfer conduit has an internal cross-sectional area substantially greater than that of the microchannel of each biochip.

In a still further embodiment, each biochip has more than one inlet port, each of which is for connection to a different liquid delivery unit.

In another embodiment of the invention, each biochip has more than one outlet port.

One particular form of biochip comprises a pair of elongate microchannels, each having at least one inlet port at its proximal end and at their distal ends connecting into a further microchannel having at least one outlet port at its distal end to form therewith a Y-shaped composite microchannel or may comprise an elongate microchannel having a bore, at least one intermediate portion of which has a different cross-sectional area to that of the rest of the microchannel or indeed may comprise a pair of elongate microchannels, each microchannel having at least one inlet port and at least one outlet port, the microchannels being connected their proximal ends and distal ends.

In one embodiment of the invention, the microchannels are all formed on one bottom face of a planar biochip sheet of translucent plastics material as open cut-out channels covered by a thin film of polymer material coated with a pressure sensitive adhesive material, the other top face of the biochip sheet mounting the input ports, the output ports and the reservoir wells which microchannels may be non-cylindrical cross-section.

With this latter embodiment of the invention, there may be provided a further open cut-out channel forming a main liquid feeder channel, the main liquid feeder channel having a liquid inlet port for connection to the liquid delivery unit and a plurality of delivery ports equal in number to the number of biochips, the liquid feeder channel being covered by a thin film of plastics material.

One particular construction of these embodiments may comprise:
an upper support plate having an upper face and a lower face in use; and
a plurality of tubes mounted in the plate and projecting proud of the faces, each tube proud of the upper face being for connection to one of the transfer conduits and at its other end for connection to one of the ports and wells.

In this latter embodiment, the releasable connection means may be provided for mounting the plate above the top face of the biochip sheet The releasable connection means may comprise:
a pair of spaced-apart columns proud of the biochip sheet and mounting a pivot bar therebetween; and
a support member pivotally mounted on the bar and having a channel-shaped elongate open mouthed slot for reception of the plate, portion of the support member forming a camming surface for engaging the top face of the biochip sheet when pivoted into a position to engage the plate above the biochip sheet.

When the biochips each have additional inlet ports and there are additional sets of main liquid feeder channels, the number of such sets equals the number of additional inlet ports for each biochip. Again, with these embodiments, the inlet ports and outlet ports on the top face have bores between entrance and exit, of substantially constant cross-sectional area and of substantially the same order of magnitude as that of the microchannels.

In one embodiment of the invention, the liquid delivery unit comprises:
a liquid outlet link assembly to provide a steady liquid delivery output rate below 10 μl per minute through the liquid delivery port of the liquid delivery unit from a link input port connected to a positive displacement pump forming part of the liquid delivery unit and having an immediate step pumping rate substantially greater than the desired steady liquid delivery output rate, the liquid outlet link assembly further comprising a hollow link body having a resistance to flow therethrough substantially less than through the liquid delivery port; and
pressure stabilising means for the link body formed by pressure compressible means connected thereto whereby, on increased pressure being encountered in the hollow link body on operation of the positive displacement pump, the pressure compressible means initially contracts to counteract the pressure rise in the liquid outlet link assembly and hence the rise in the liquid flow rate through the liquid delivery port and then as delivery of liquid takes place through the liquid delivery port expands to maintain the pressure within the liquid link assembly relatively stable.

In this latter embodiment, the pressure compressible means may comprise a gas bubble or may comprise more than one gas bubble and the aggregate volume of the bubbles is a multiple of the volume of liquid dispensed in one step of the pump. With these two latter embodiments, the aggregate volume of the gas bubble or bubbles is significantly larger than the volume of the liquid dispensed in one step of the pump.

In another embodiment, the aggregate volume of the gas bubble or bubbles is comparable to the volume of the pump, which may be in the range of 10 to 100 microlitres.

In another embodiment of the invention, the compressible means comprises an elastic membrane forming part of the link body or may comprise expandable tubing which forms the expansion means.

Ideally, control means is provided and is connected to a flow conditions sensing means for the liquid outlet link assembly for causing the pump to operate to provide the desired flow rate through the outlet port.

In one embodiment, the flow conditions sensing means is a pressure sensor connected to the link body or can be an optical flow sensing assembly such as a camera.

In one embodiment of the invention, the pump is a syringe pump.

In another embodiment, the volume pumped for each step of the syringe pump is of the order of 0.2 μl.

Further, the invention provides a cell based assay assembly comprising a biochip assembly as described above and detection and recording equipment for conducting an assay on a biological cell as it is delivered through the biochip assembly. The detection and recording equipment may comprise an optically inverted microscope, a digital camera and computerised recording, monitoring and control means. In another embodiment of the invention, it may comprise an epifluorescence device.

Further, the invention provides a method of conducting a biological cell assay on a cell based assay assembly as described above comprising the steps of:
(a) connecting the liquid delivery outlet port to a well by a transfer conduit;
(b) aspirating liquid from the well into the transfer conduit;
(c) connecting the transfer conduit to an inlet port;
(d) delivering liquid from the transfer conduit through the biochip and then repeating steps (a) to (d) as often as required; and
(e) then carrying out the assay with the detection and recording equipment as the final step (d) is being carried out.

In this latter method, the additional step, after one or more of step (d), is carried out of simultaneously using another transfer conduit to connect the outlet port of the biochip to another well.

With this method, when the biochip is manufactured using a thin film of polymer coated with a pressure sensitive adhesive material, the additional step is performed, after the assay has been completed, of removing the film and carrying out further tests on the biological cells adhering to the film.

In this method, the additional step may be performed of filling the transfer conduit with the system liquid.

Further, the step may be performed of replacing the transfer conduit between aspirating liquids from wells during steps (a)-(d) in order to avoid cross-contamination. Further, after aspirating liquid from a well, the additional step of flushing system liquid through the transfer conduit is carried out.

Further, in the method as described above, there is provided a method in which a desired flow rate within the biochip assembly is achieved by:
determining the required pressure within the liquid delivery unit to achieve the desired flow rate by first determining a flow rate for the pump which maintains a constant pressure within the biochip assembly to provide a fluidic resistance factor for each biochip determined by dividing the pressure by the flow rate and then multiplying the desired flow rate by this fluidic resistance factor to provide the required pressure; and
then operating the pump to provide the required pressure.

When in this latter method, the pressure drops below the required pressure by a predetermined amount the pump is operated to deliver liquid into the liquid delivery unit and when the require pressure is exceeded by a predetermined amount, the pump is reversed to aspirate liquid.

Alternatively, the flow rate of the pump may be varied to maintain the pressure within a predetermined range of pressure.

Further, the required pressure may be achieved with a predetermined displacement volume of the pump over a predetermined time by varying the compressibility of the pressure compressible means.

In one embodiment of the invention, the varying of the compressibility of the pressure compressible means comprises adding or reducing the amount of gas within the link body.

### DETAILED DESCRIPTION OF THE INVENTION

The invention will be more clearly understood from the following description of some embodiments thereof, given by way of example only, with reference to the accompanying drawings, in which:

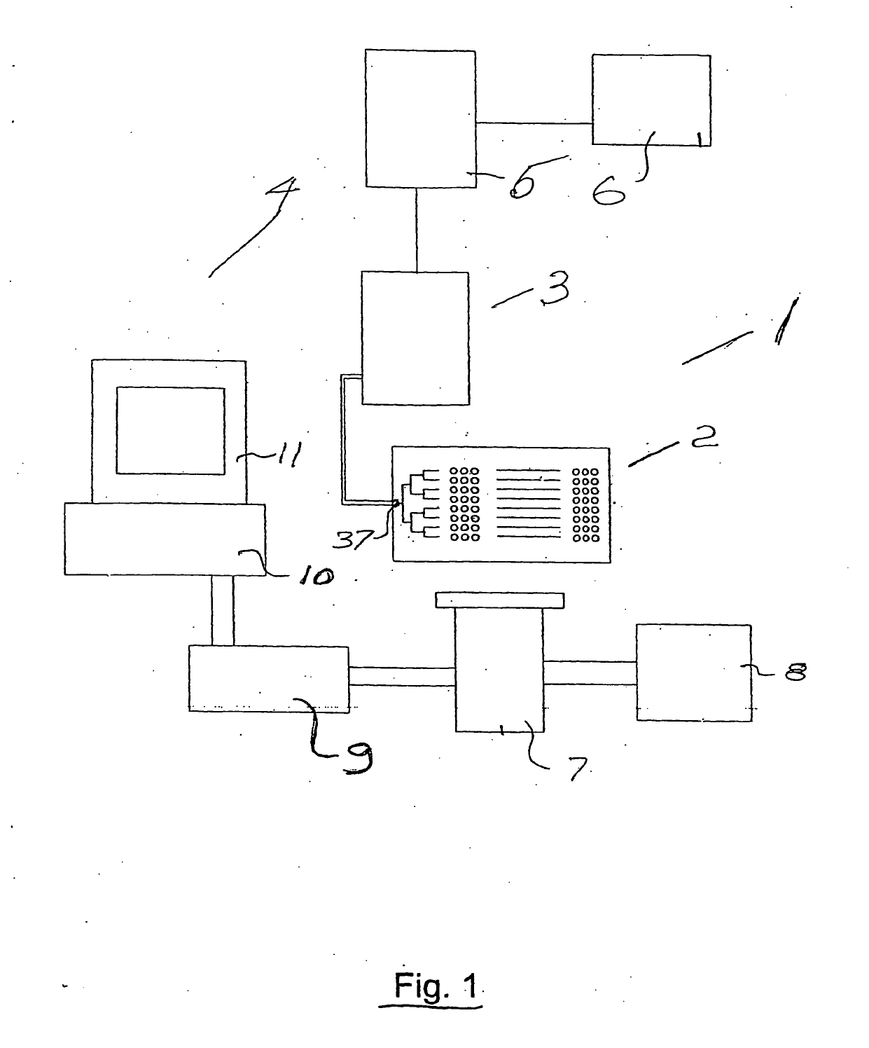

FIG. 1 is a diagrammatic layout of a cell based assay assembly according to the invention,

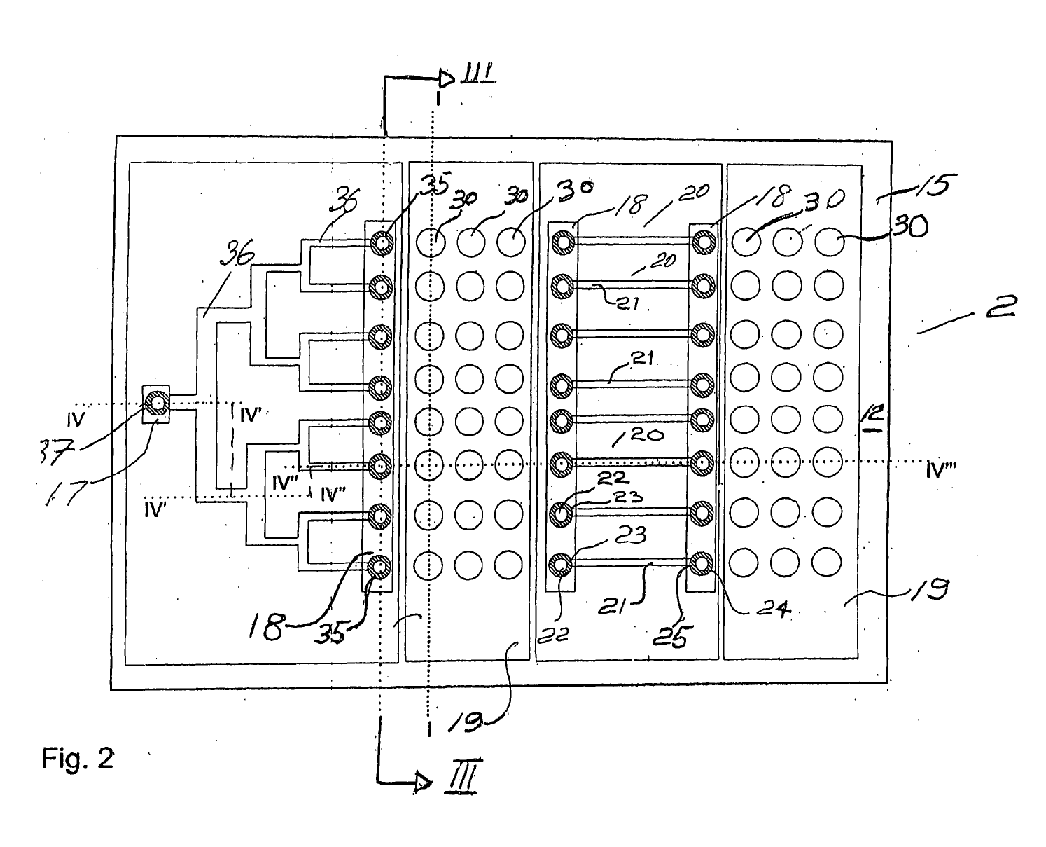

FIG. 2 is a plan view of biochip assembly used in the assay assembly of FIG. 1,

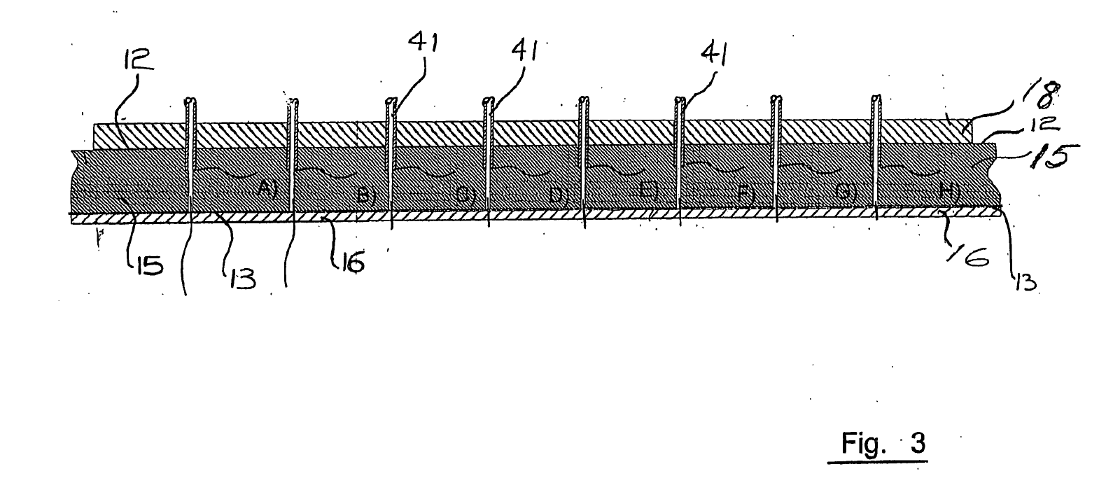

FIG. 3 is a sectional view along the lines III-III of FIG. 2,

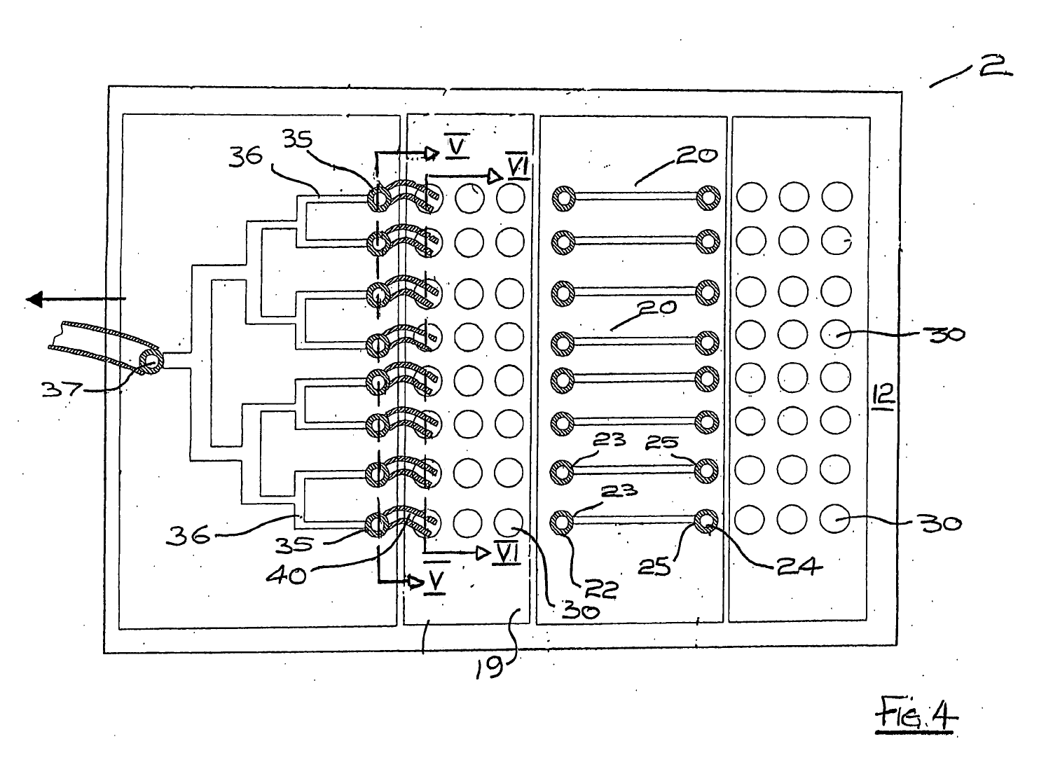

FIG. 4 is a plan view similar to FIG. 2 showing the biochip assembly in another position of use.

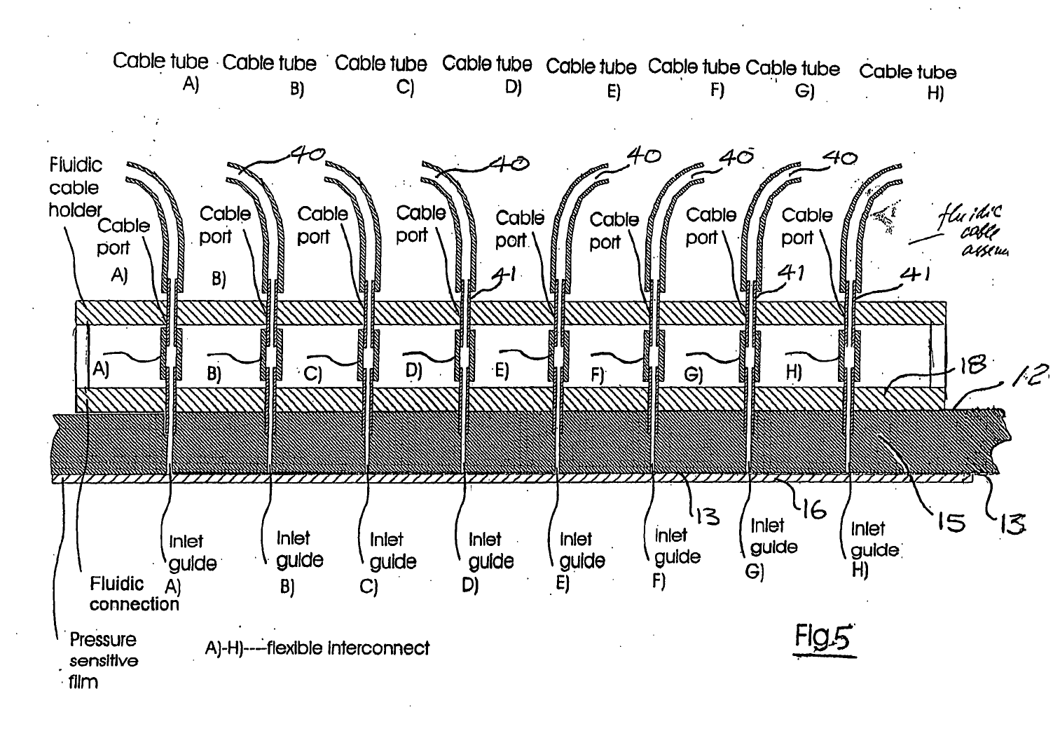

FIG. 5 is a sectional view along the lines IV-IV of FIG. 4,

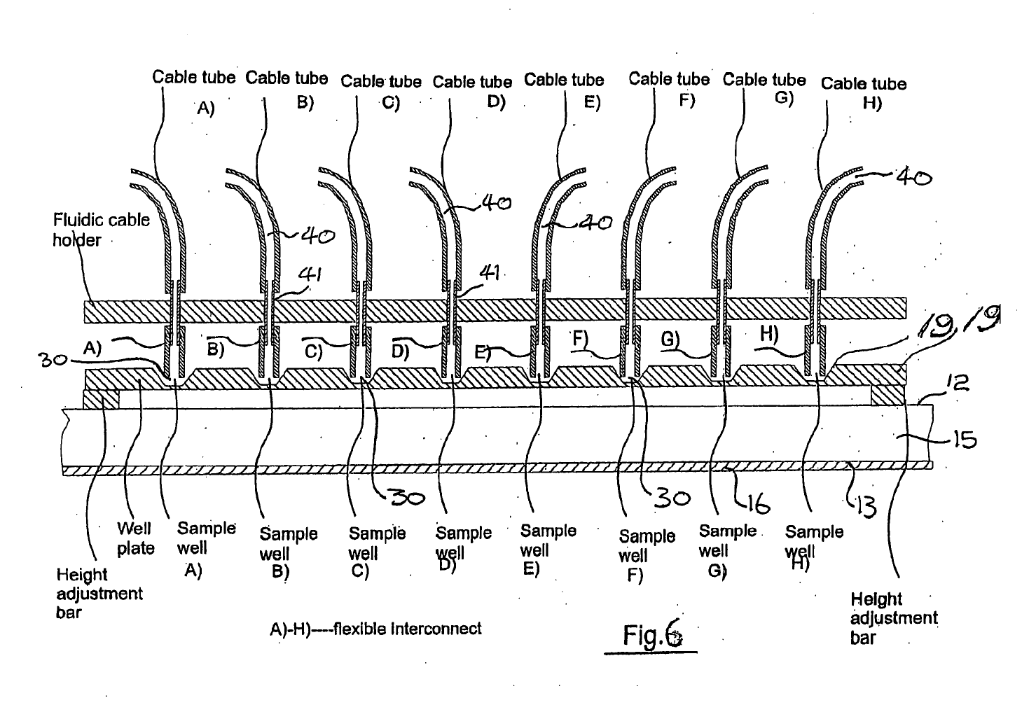

FIG. 6 is a sectional view along the lines VI-VI of FIG. 4,

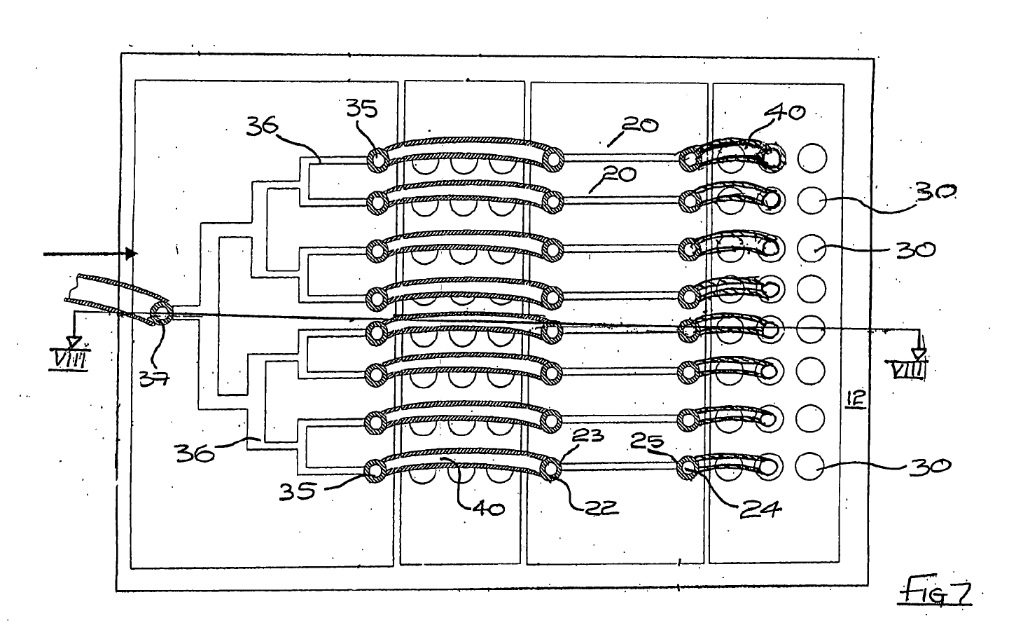

FIG. 7 is a plan view similar to FIG. 2 showing the biochip assembly in another position of use,

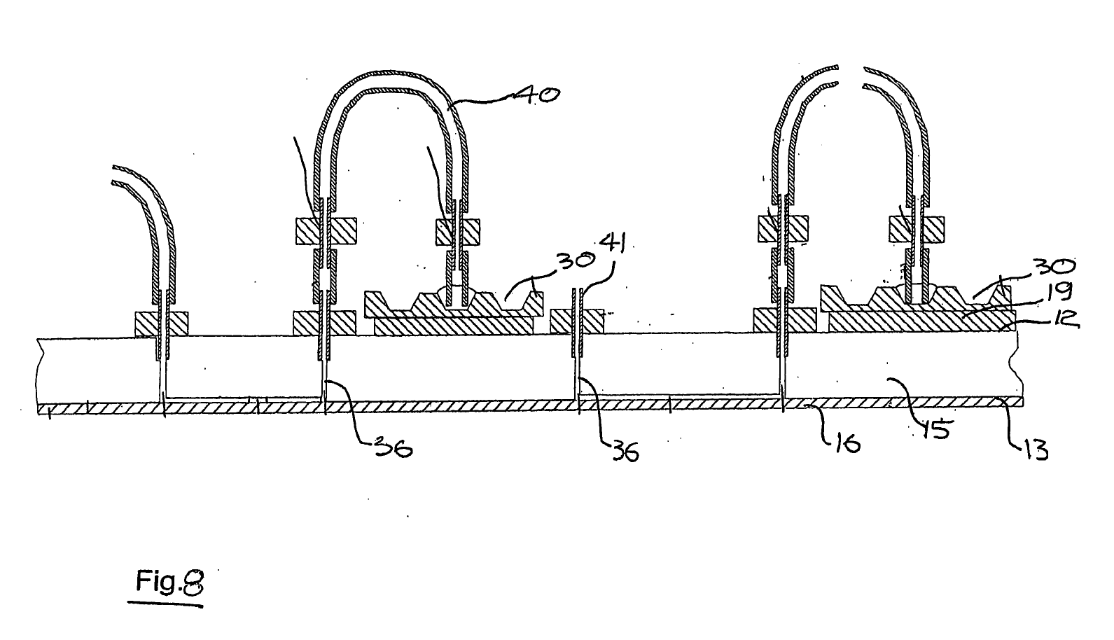

FIG. 8 is a sectional view along the line VIII-VIII of FIG. 7,

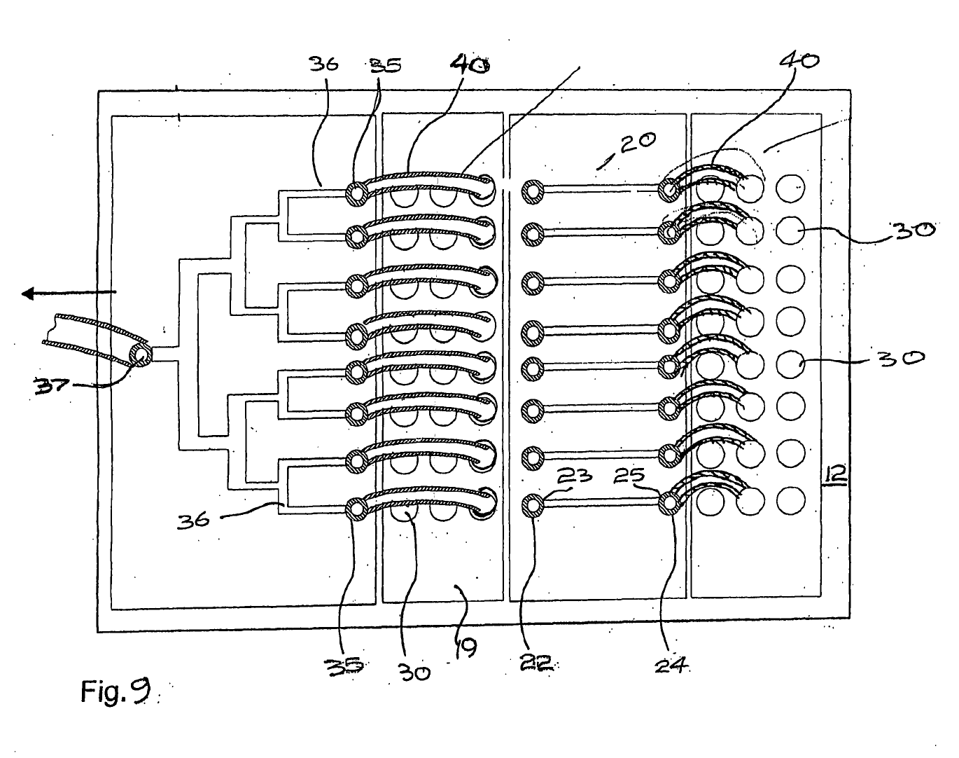

FIG. 9 is a plan view, similar to FIG. 2, showing the biochip assembly in another position of use,

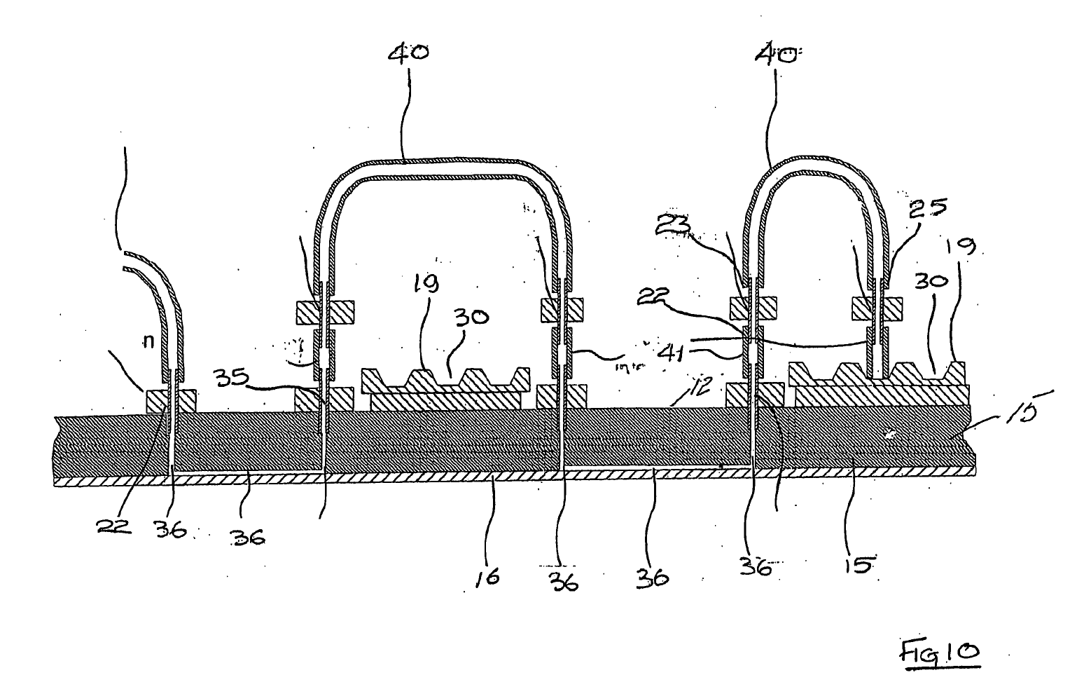

FIG. 10 is a sectional view along the lines X-X of FIG. 9,

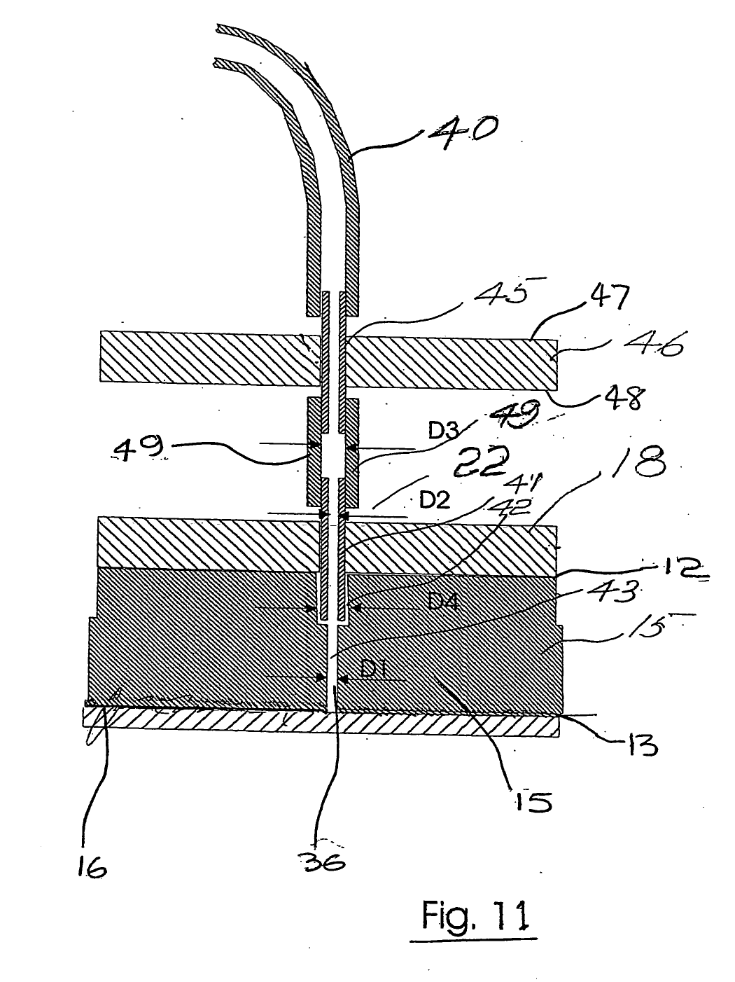

FIG. 11 is an enlarged sectional view through portion of the biochip assembly of FIG. 2,

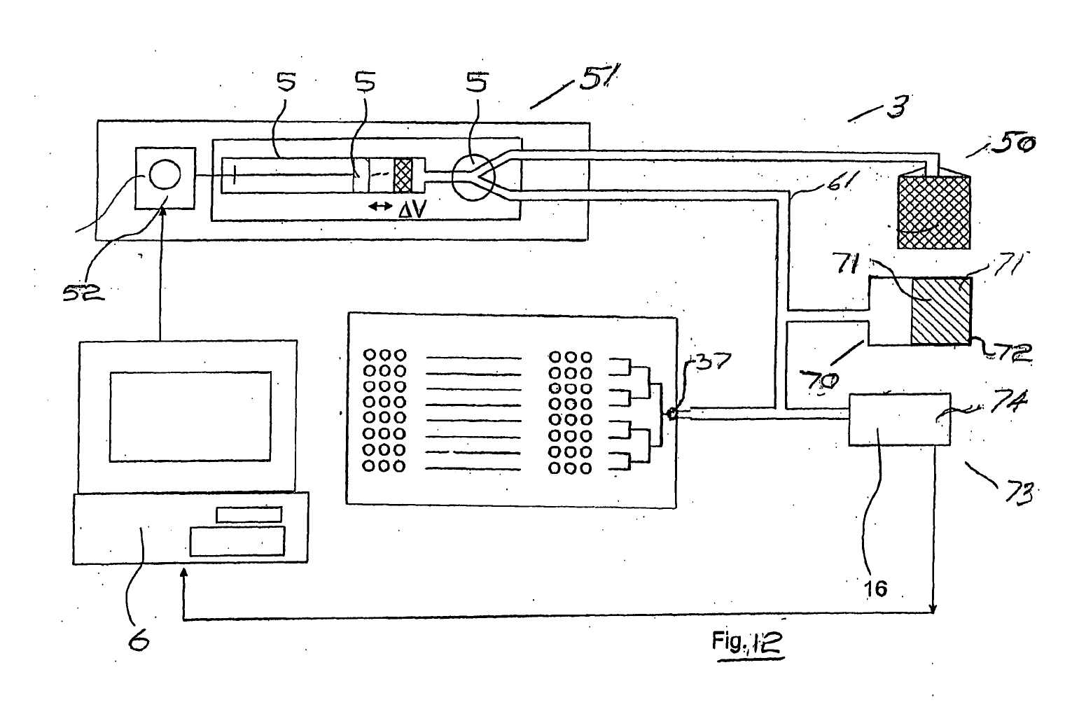

FIG. 12 is a diagrammatic view of a liquid delivery unit forming part of the cell based assay assembly according to the invention,

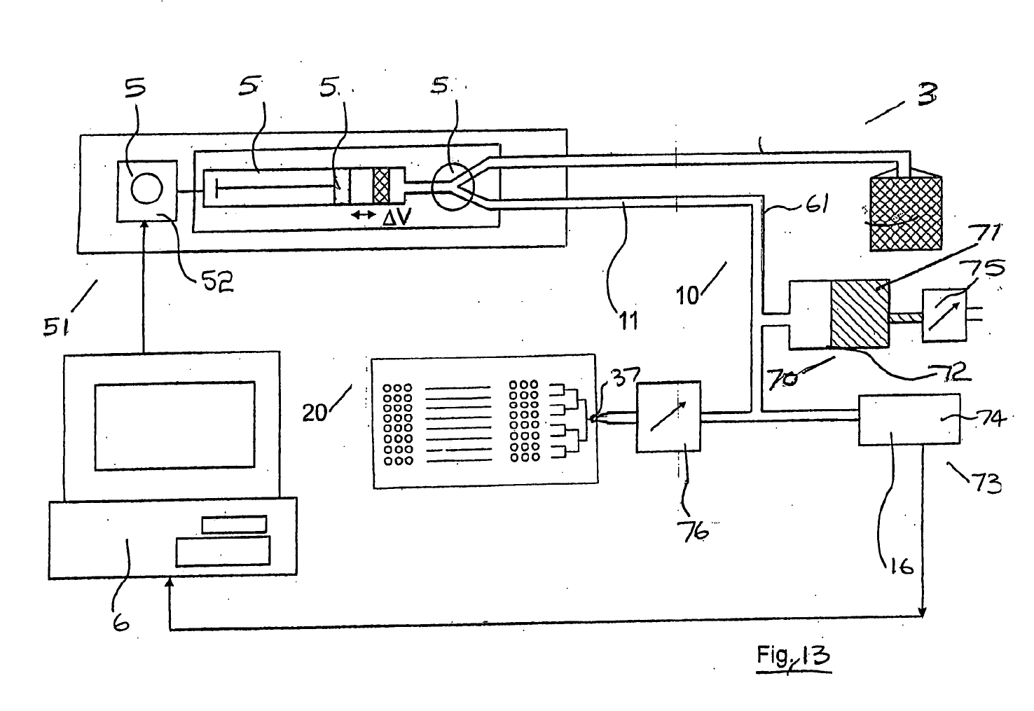

FIG. 13 is a view similar to FIG. 12 of another construction of liquid delivery unit,

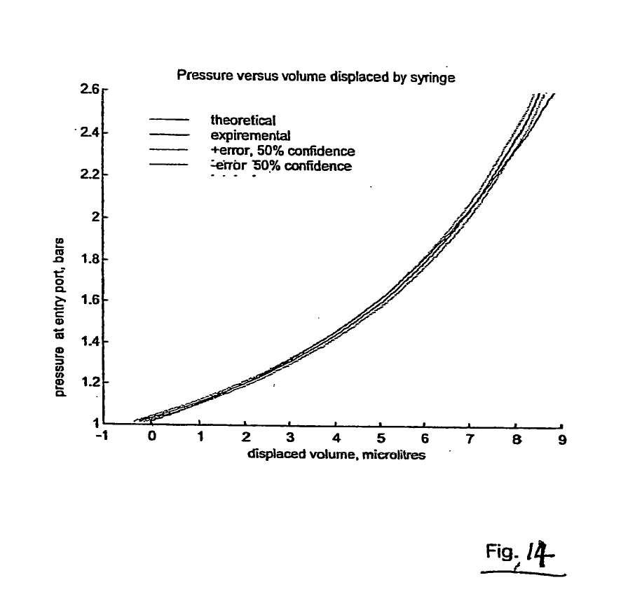

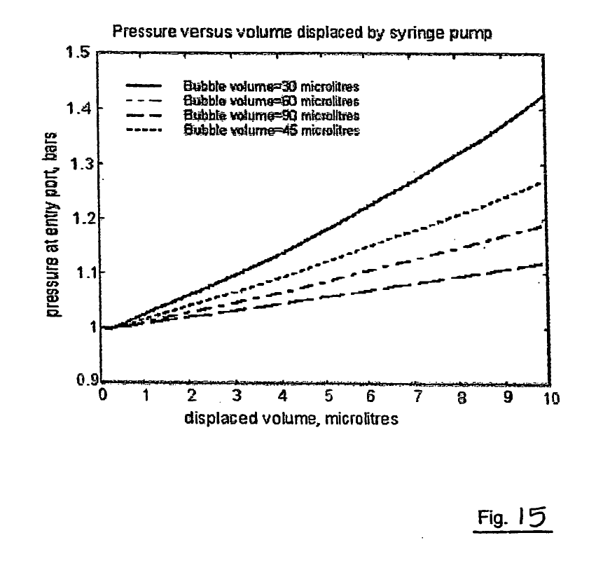

FIGS. 14 and 15 are graphs showing results of tests carried out on the liquid delivery unit according to the invention,

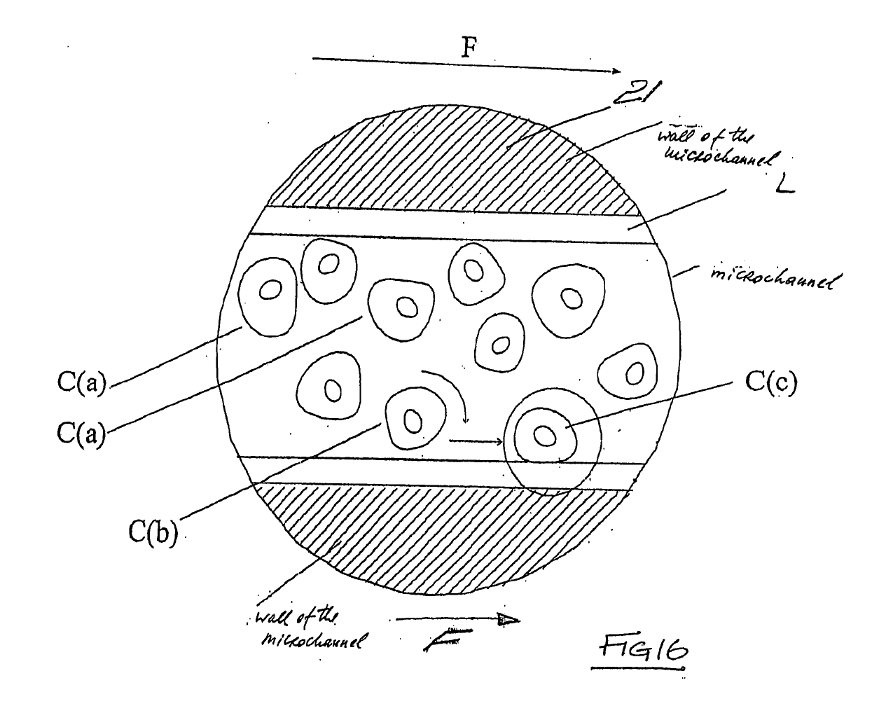

FIGS. 16 to 19 are enlarged views of portions of microchannels forming part of a biochip assembly according to the invention illustrating assays being carried out,

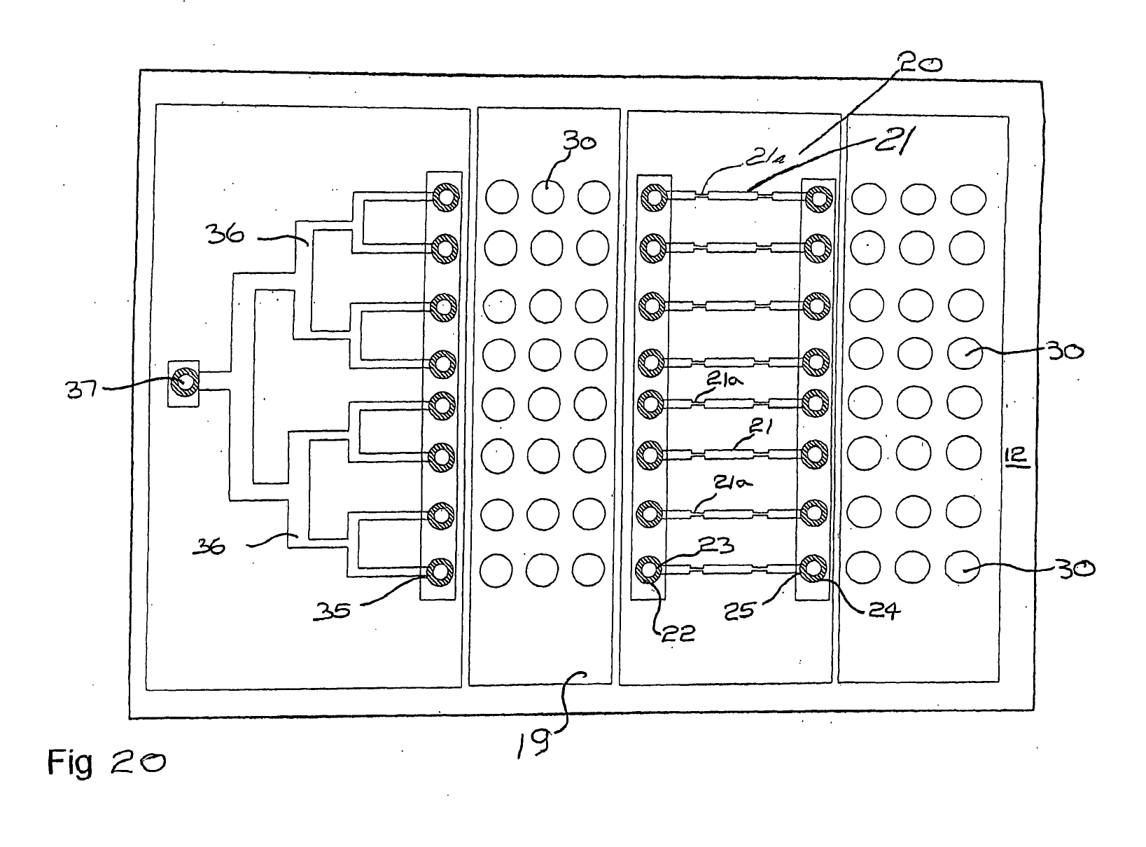

FIG. 20 is a plan view, similar to FIG. 2, of an alternative construction of biochip assembly according to the invention,

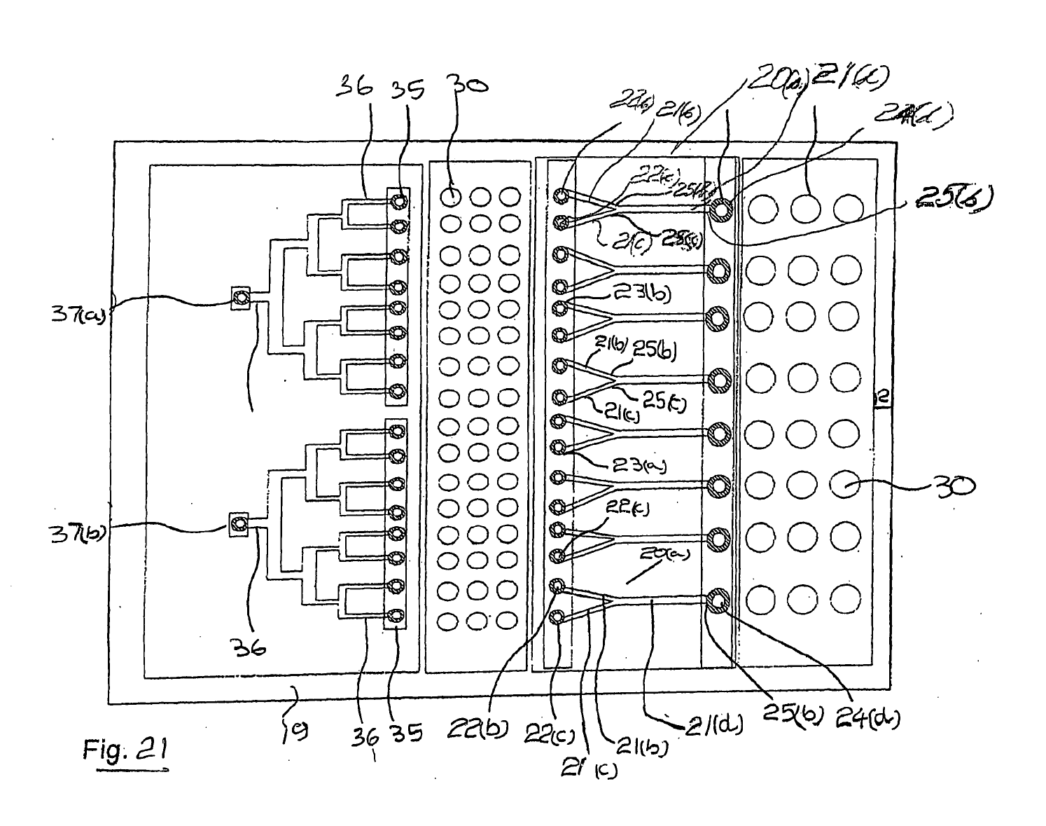

FIGS. 21 to 23 are plan views similar to FIG. 2 of another construction of biochip assembly in various positions of use,

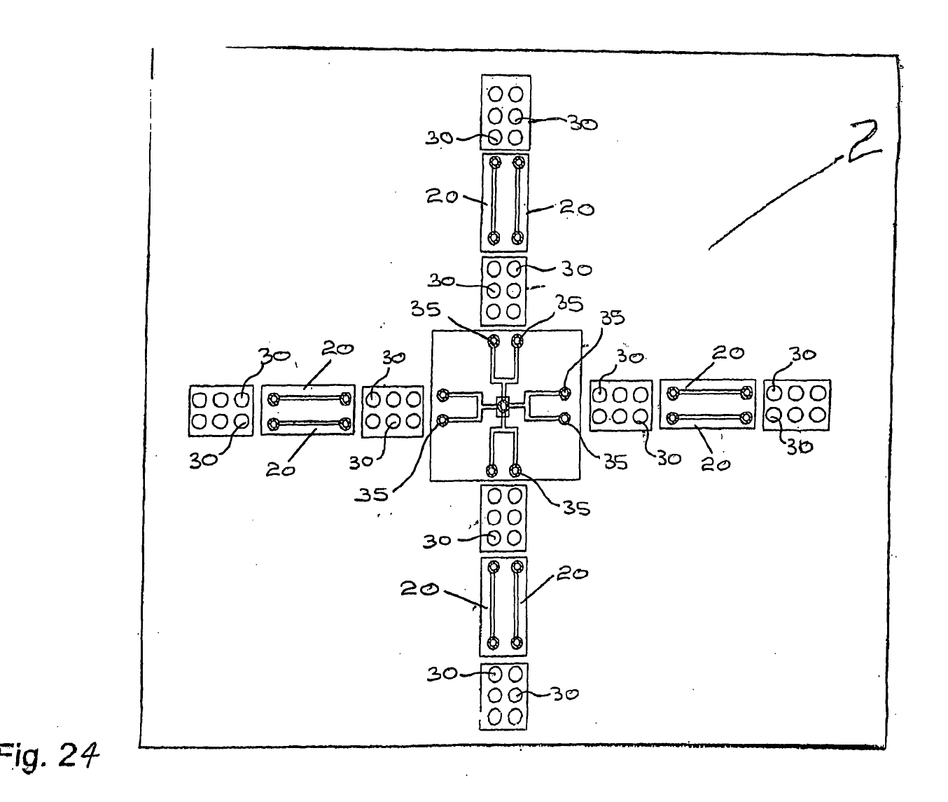

FIG. 24 is a plan view of a still further construction of biochip assembly,

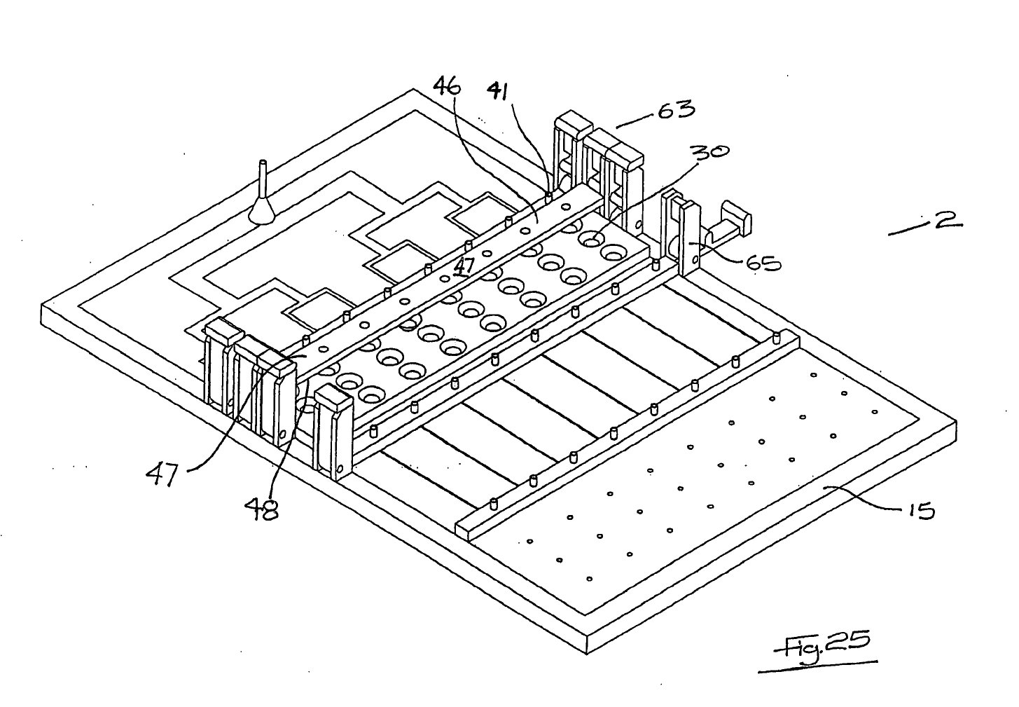

FIG. 25 is a perspective view of a biochip assembly according to the invention,

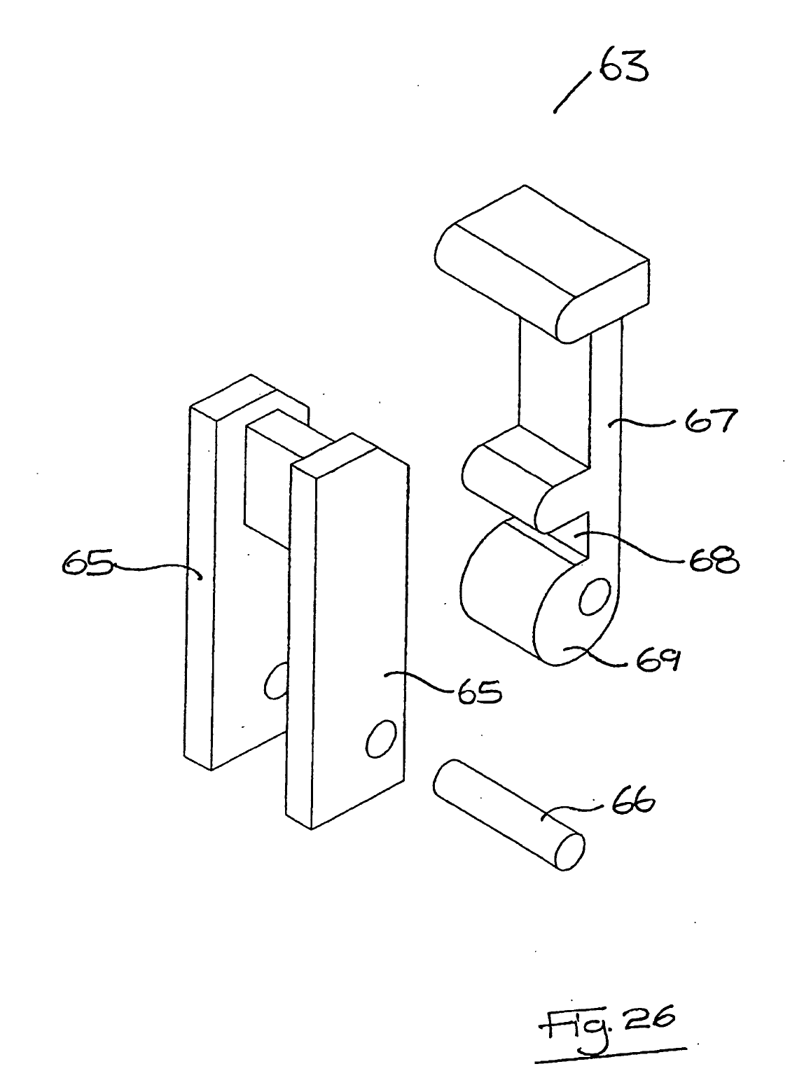

FIG. 26 is an exploded view of portion of the biochip of FIG. 25,

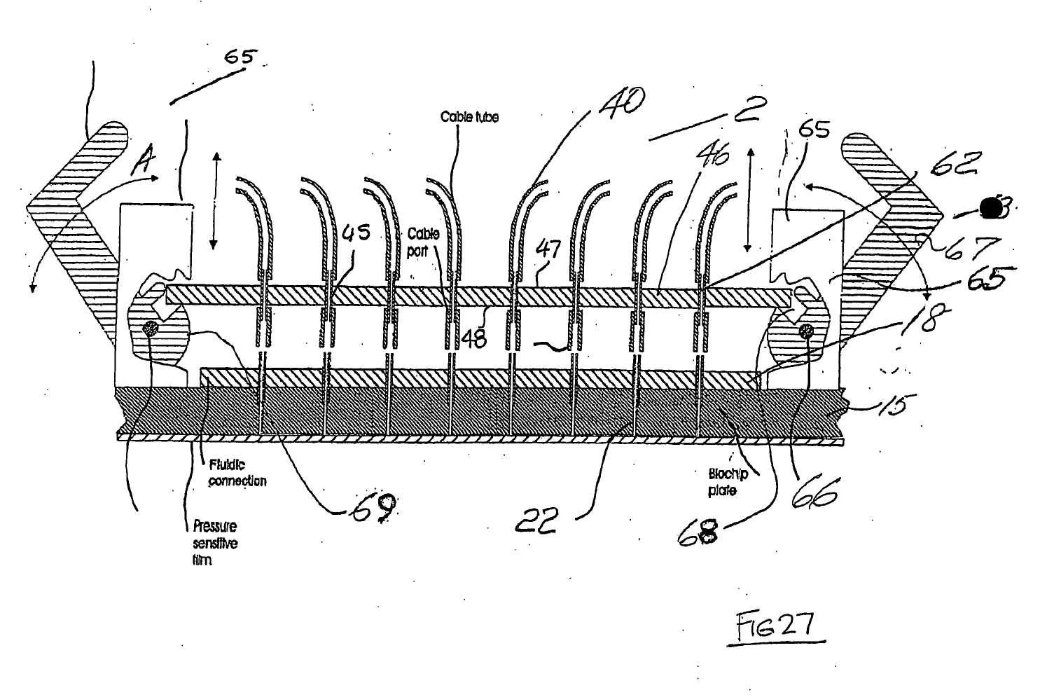

FIG. 27 is a typical sectional view through the biochip assembly of FIG. 25,

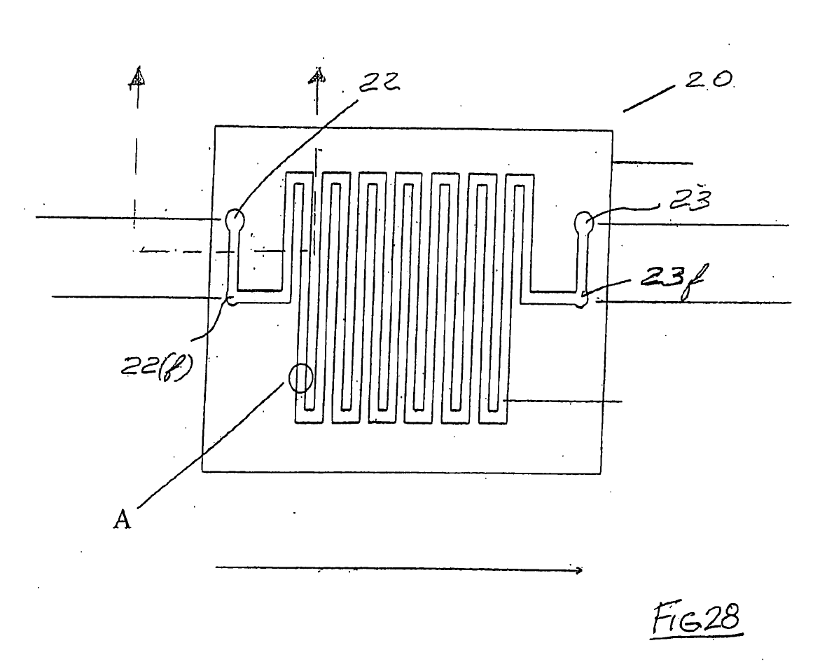

FIG. 28 is a plan view of a construction of biochip forming part of a biochip assembly according to the invention, and

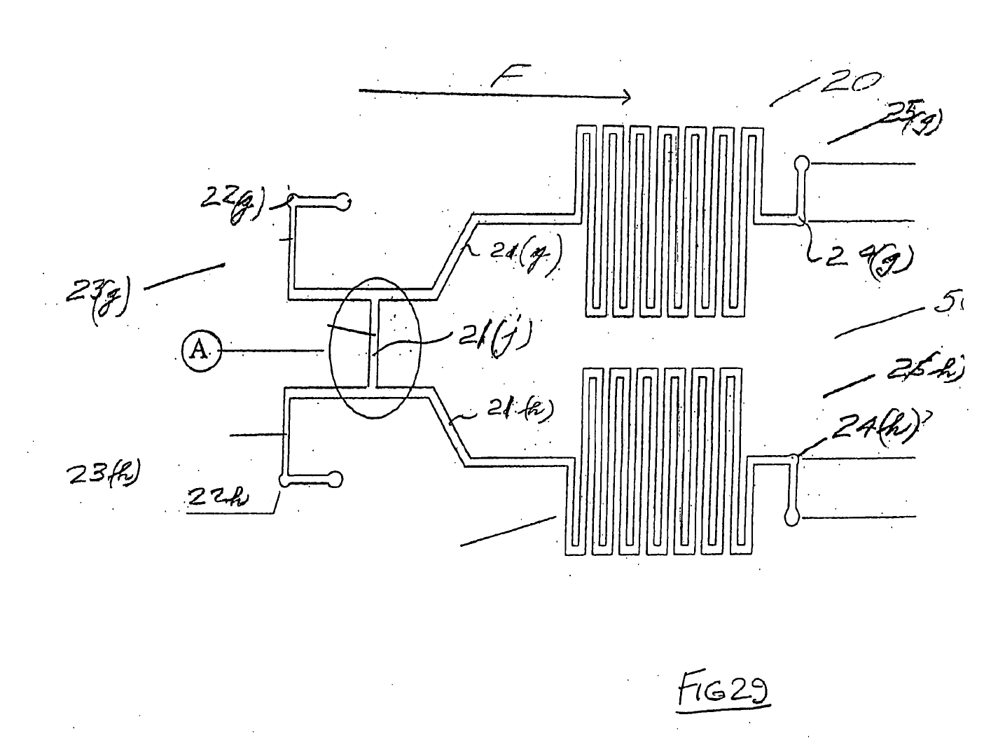

FIG. 29 is a plan view of a still further construction of biochip.

Referring to the drawings and initially to FIG. 1, there is illustrated a cell based assay assembly, indicated generally by the reference numeral 1. The cell based assay assembly 1 comprises a biochip assembly 2 connected to a liquid delivery unit 3 and detection and recording equipment, indicated generally by the reference numeral 4. Further, there is provided control means, some of which is provided by a pump controller 5 connected to the liquid delivery unit 3 and a computer 6. There is provided computerised recording, monitoring and control means so that the biochip assembly 1 and the detection and recording equipment 4 operate in the desired manner. It does not require description as there are so many ways of carrying it out once the functions required are stated. Strictly speaking, the computer 6 forms part of the pump controller 5. An optically inverted microscope 7, connected to an epifluorescence device 8 and to a digital camera 9, forms part of the detection and recording means 4. The digital camera 9 is in turn connected to a recorder 10 having a monitor 11, all of which comprises part of the detection and recording equipment 4.

Referring now to FIGS. 2 to 11 inclusive, there is described many features of the biochip assembly 2, however, certain structural features are not shown in this embodiment as it would simply confuse the issue. They are described later with reference to FIGS. 25 and 26.

Further, since the biochip assembly 2 is manufactured from an optically transparent plastics material, features on both the top and bottom of the biochip assembly 2 can be seen in plan view, however, it unnecessarily confuses the description again to distinguish between those parts of the biochip assembly that are on the top face of the sheet of plastics materialand those on the bottom face. It will be apparent from the remainder of the drawings which parts are on the top and which ones are on the bottom. In any case, the location of the parts is irrelevant to the understand of the invention. The biochip assembly 2 essentially comprises a biochip or planar sheet 15 having formed in a top face 12 and bottom face 13 thereof, various parts or features and the bottom face 13 is covered by a plastics film 16, in this embodiment, polymer coated with a pressure sensitive adhesive. Various support plates, 17, 18 for various ports, and 19 for aspiration wells are provided and are mounted above the biochip sheet 15, in conventional manner. These are not described in more detail beyond being necessarily mechanical arrangements.

The biochip assembly 2 comprises a plurality of biochips, each indicated generally by the reference numeral 20 and each comprising an elongate microchannel 21 shown as a relatively short microchannel in FIG. 2, having an inlet port 22 mounted adjacent its proximal end 23 and an outlet port 24 mounted adjacent its distal end 25. There is provided at least one separate reservoir well 30, in this embodiment, six reservoir wells 30 for each biochip 20, one set of three reservoir wells 30 adjacent each inlet port 22 and another set of three reservoir wells 30 adjacent the outlet port 24. A minimum of one well 30 is required for each inlet port 22 while practically at least two are necessary. A well 30 adjacent the outlet port 24 could be used with the inlet port 22 and vice versa. The wells 30 are not fluidically coupled to the ports 22 and 24, except as described below. Thus, there is no permanent fluidic connection. In this embodiment, there is provided a liquid delivery port 35 for each biochip 20 and this port 35 is connected by a set of main liquid feeder channels 36 to a liquid inlet port 37 for connection to the liquid delivery unit 3, as will be described in more detail later.

A plurality of removable separate enclosed transfer conduits 40 are provided and are provided by lengths of flexible plastic tubing.

The reservoir wells 30 are essentially conventional microwells in the plate 19, as can be seen from FIG. 6. Typical volume of the reservoir wells 30 is some 1 to 50 microlitres, although values outside this range are also possible.

Support plates and releasable connection means for the transfer conduits 40 are provided and in some cases, parts of them are shown in FIGS. 1 to 11, however, for simplicity, they are not described in any detail but are mainly referred to in passing.

Referring now specifically to FIG. 11, there is shown in more detail one of the inlet ports 22. The inlet port 22 comprises a tube 41 securely mounted within the support plate 18, which tube 41 projects into a hole 42 having a diameter D4 in the biochip sheet 15 which in turn extends into a bored hole 43 having a diameter D1. The diameter D1 is chosen so that the cross section of the bored hole 43 is comparable to that of the microchannel 21 to which it forms the inlet port. The microchannel 21 extends orthogonally from the hole 43. Mounted above the support plate 18 and in spaced relation thereto is an upper support plate 46 having a top face 47 and a lower face 48. The support plate 46 carries rigid tubes 45, each proud of the top face 47 for connection to one of the transfer conduits 40. The tube 45 also projects below the lower face 48 to connect to the tube 41 of the inlet port 22 by a further length of flexible interconnect tube 49. While the inner diameter D3 of the interconnect tube 49 is greater than the inner diameter D2 of the tube 41, the diameter D2 is maintained as closely as possible to that of the bore 43. It is important that the diameter D4 is made as small as possible so that the tube 41 forms a force-fit therein. While it is more difficult to force fit the tube 41 within the biochip plate 15, it has been found that air bubbles do not form within the liquid being transferred, nor indeed do blockages occur. This is somewhat contrary to what one would normally expect.

Before describing in some detail the construction and operation of the delivery unit and the various assays that may be carried out in accordance with the cell based assay assembly 1 according to the invention, it is advantageous to describe the use and operation of the biochip assembly 2.

In operation, for example, when conducting a cell adhesion study, different ligands could be provided in each one of the eight wells 30 for each of the biochips 20 being deposited there in conventional manner such as by pipetting. Similarly, the same sample containing suspension of cells could be placed in one of the eight wells 30 for each of the biochips 20, obviously both adjacent the input ports 22. Typically, the channels 36 and conduit 40 are filled up with a system liquid, e.g. distilled water or PBS. The system liquid fills up the conduit 40 and extends up to the front end of the tube 45. Alternatively, the system liquid terminates a short distance away from the end of the tube 45 so that an air bubble could be formed between the system liquid and the sample liquid to reduce the chances of cross-contamination. Then, the transfer conduit 40 is connected between each liquid delivery ports 35 and well 30 of the same biochip and ligand is aspirated into the transfer conduit 40 as illustrated in FIGS. 4, 5 and 6. Then, the transfer conduit 40 is connected between the liquid delivery ports 35 and the inlet ports 22 (see FIG. 7). Also, the outlet port 24 is connected to one of the wells 30 adjacent the output ports 24. This latter step is not essential. Then, ligand is delivered through the transfer conduits 40 into each biochip 20 with surplus ligand being delivered out the outlet ports 24 into the appropriate well 30. The conduits 40 are then connected between the wells 30 containing the cell samples and the same operation as with the ligands is used to draw the cell samples into the transfer conduits 40 (FIGS. 8 and 9). Then the transfer conduits 40 are connected to the inlet ports 22 again. The cells are delivered through the biochips 20 for the assay to take place (see FIG. 10). If cross-contamination through conduits 40 is critically unacceptable, they can be disposed or retained for cleaning between the subsequent steps of drawing the liquid into them. However, in many cases, It may be sufficient to expel some system liquid from conduits that was cross-contaminated by the ligand or cell suspension by diffusion in the conduit.

Referring now to FIGS. 12 to 15, there is illustrated two embodiments of the liquid delivery unit 3 which comprises a liquid outlet link assembly, indicated generally by the reference numeral 50, to provide a steady liquid delivery output rate below 10 μl per minute through the liquid inlet port 37. The liquid outlet link assembly 50 is connected to a positive displacement pump, indicated generally by the reference numeral 51, which forms part of the liquid delivery unit 3. The positive displacement pump 51 has an intermediate pumping rate substantially greater than the desired steady liquid delivery output rate. The positive displacement pump 51 is a syringe pump operated by a stepper motor 52. The syringe pump 51 has a plunger 53 mounted within a syringe body 54. One incremental step of the plunger 53 causes the plunger 53 to displace a volume ΔV. The position of the plunger 53 in the new position is shown by the cross-hatched lines. The pump 51 feeds a valve 56 which connects the pump 51 to the liquid outlet link assembly 50 which comprises a hollow link body 61. The valve 56 essentially forms a link input port and is identified by the same reference numeral. Reference herein to the link input port is a reference to the valve and vice versa. The hollow link body 61 has a resistance to flow which is substantially less than that through the liquid inlet port 37.

A pressure stabilising means, indicated generally by the reference numeral 70, comprises pressure compressible means, in this embodiment, an air bubble, identified by the reference numeral 71, within a reservoir 72. A flow condition sensing means, indicated generally by the reference numeral 73, is provided which can comprise, as it does in this embodiment, a pressure sensor 74. The computers previously described form control means when linked to the condition sensing means 73. Any suitable gas could equally be used instead of air. The pressure sensor 74 is connected to the hollow link body 61.

It will be appreciated that the resistance to flow at the liquid inlet port 37 will be substantially greater than the resistance to flow through the hollow link body 61.

In operation, when the positive displacement pump 51 operates, there will be an immediate increase in pressure at the valve 56 which is effectively an immediate increase in pressure at the liquid inlet to the hollow link body 61. This increase in pressure in the hollow link body 61 will cause the pressure compressible means 71, namely, the air bubble 71, to contract This will immediately counteract the pressure rise in the hollow link body 61. This in turn means that, after an initial rise, there will be an immediate drop in pressure at the liquid inlet port 37 and thus the rise in liquid flow rate is reduced. Effectively, therefore, a steady pressure between well defined limits will be exerted at all times at the liquid inlet port 37. As can be seen from the previous drawings, the flow will then be split into a number of separate channels forming part of the main liquid feeder channels 36 to each of the liquid delivery port 35.

Essentially, therefore, what is provided is a liquid outlet link assembly which supplies a steady liquid delivery output rate, usually (between 100 pl/min and 10 ul/min) below 10 μl per minute through the liquid inlet port 37. Also, because the computer 6 and hence the controller 5 are connected to the pressure sensor 74, the operation of the positive displacement pump 51 and hence the liquid outlet link assembly 50, may be controlled.

Before discussing the operation in any more detail, it is worth discussing briefly, the method of pumping according to the present invention. Essentially, the system comprises three distinct units, namely, the positive displacement pump which operates in a series of steps. This in turn feeds through what is effectively a liquid outlet link assembly having the pressure stabilising means which in turn feeds the elongate enclosed biochip assembly 2 from the liquid inlet port 37. What the bubble does is that it adds expandability and compressibility to the pumping system which allows accurate regulation of pressure at the liquid inlet port 37. It will be appreciated that this is contrary to conventional methods where considerable efforts are taken to avoiding and removing air bubbles. One could expect that an expandable inner volume would compromise the dispensing accuracy of the pump and lead to error.

However, this is not the case. As is known, the velocity ν of the liquid in a circular capillary under a limitation of laminar flow is subject to Poiselle's law,

$$ν = \frac{( {p}_{1} - {p}_{2} ) * {r}^{2}}{8 * η * L} ,$$

where P₁, P₂ are pressure values at the inlet and outlet of the capillary, r is the radius of the capillary, η is the viscosity of the fluid, L is the length of the capillary and * indicates multiplication.

The embodiment described above uses a positive displacement pump in combination with this expandable/compressible element formed by the air bubble to produce a small pressure difference between the inlet port 22 and outlet port 24 of each biochip 20 and therefore to establish slow movement of the liquid inside the microchannel structure. Once this pressure difference is established in each case the resulting velocity of the liquid would depend on the viscosity of the liquid, diameter and length of the microchannel structure according to Poiselle's Law. For example, for a capillary with a. diameter of 50 μm and a length of 20 cm, 5-mbar pressure gradient will create water flow with mean velocity of about 75 μm/s.

Suppose, the initial volume of the gas bubble is V₀. Suppose then the plunger of the syringe pump moves and expels a volume of liquid ΔV. If the liquid is enclosed in unexpandable conduit and the liquid is practically uncompressible, the volume of the air bubble will decrease by essentially the same amount ΔV. At this point, we have made the assumption that the liquid is enclosed, therefore the liquid outlet is closed and the liquid cannot exit it. This causes an increase in pressure that can be calculated from gas state law, PV=RT, namely, pV=const:
p₀*V₀=p_{N}*(V₀−ΔV),
p_{N}=p₀+Δp,
where p_{N} is the pressure on the air bubble after the movement of the plunger and p₀ is the pressure before the movement

Thus

$$\frac{Δ p}{{p}_{0}} = \frac{Δ V}{{V}_{0} - Δ V} . \qquad ( 1 )$$

For example if the volume of the bubble is halved: ΔV=V₀/2, the pressure will increase by a factor of two. The ratio of the initial volume of the bubble to the smallest displaced volume within the syringe pump gives the accuracy of building up the pressure at the entry port. The greater is the initial volume of the bubble the higher is the accuracy of the pressure regulation.

In practice the system is not enclosed and is connected to the biochips 20, that is to say, the microcapillary or microstructure. In this case there will be a flow of liquid through the microchannel structures which will cause the volume of the bubble to gradually return to the initial state. However if the volume of the air bubble is several orders of magnitude greater than the volumetric flow rate through the microstructure multiplied by the time of the experiment, the change in the volume of the bubble will be negligible and therefore the pressure at the entry port will be practically constant. In the case when the flow of the liquid through the microchannel structure is causing significant change in pressure, the pressure can be corrected by displacing additional volume of liquid from the syringe pump. Alternatively, for such a case the volume of the air bubble can be increased.

Referring now to FIG. 13, there is shown an alternative construction of liquid delivery unit according to the present invention, substantially similar to that of FIG. 1 and again identified by the reference numeral 3, in which other parts similar to those described with reference to FIG. 12 are identified by the same reference numerals. In this embodiment, the air reservoir 72 is provided with an air reservoir valve 75 and the hollow link body 61 has a control valve 76 adjacent the liquid delivery port 37. The valves 75 and 76 are connected to the computer 6.

To compare the ratio between the velocity of the liquid in a system without one containing an air bubble and with one containing an air bubble, initially there was no air bubble in the system, that is to say, the pressure activated expansion means was disconnected and a 100 steps of displacement were applied to the syringe plunger which corresponded to a total displaced volume of 0.2 μl over a time period of 1 second. Under these conditions, the mean velocity of the liquid in the microchannel assembly 21 having a diameter of 10 μm and a length of 20 cm was calculated as follows:
ν=V/St
where ν=velocity, V=volume of the liquid expelled by the plunger, S=area of the capillary, t=time taken to expel the liquid.

$$ν = V \text{/} St = \frac{0.2 μ l}{π * {( 50 μm )}^{2} \text{/} 4 * 1 sec} \approx 10 cm \text{/} sec$$

10 cm/sec is the linear velocity of liquid in a system without pressure compressible means.

When measured with an air bubble in a microchannel structure with a diameter of 50 μm and a length of 20 cm with an air bubble of 40 μl, the velocity of liquid was only 50 μm/sec. Therefore, the ratio R of velocity in the two systems was

$$R = \frac{10 cm \text{/} sec}{50 μm \text{/} sec} = 2000$$

Thus, in accordance with the present invention, it was possible to achieve a velocity of 2000 times lower and a better flow regulation than with the conventional use of a stepper motor. Obviously, if the diameter of the channel of the microchannel assembly is reduced, this will even further increase the velocity in a conventional system without pressure compressible means. On the other hand, the velocity in the system with pressure compressible means according to the present invention will decrease according to Poiselle's law and hence this ratio R will increase. If, however, the reverse takes place, then the ratio R will decrease. Similarly, should the length of the microchannel structure be increased, this will increase the resistance and hence increase the ratio R as the velocity in the microchannel structure will decrease in the system according to the present invention. If, however, the microchannel structure were to be a short channel with large cross section, then there would be no great advantage in using a bubble of air.

We can see from this analysis that the advantage of using pressure compressible means becomes significant when dealing with microfluidic structures. For capillaries with relatively large cross section, pressure compressible means only adds to the error of volume dispensing. An additional advantage of using pressure compressible means is that it dampens pressure surges. In order to achieve the calculated 10 cm/sec velocity, the large excess pressure must be created at the input of the capillary. Such large pressure surges can be detrimental to certain biological liquids, e.g. cell suspensions. As the flow velocity is reduced according to the example by a factor of 2000 by means of air bubble, the excess pressure is also reduced by the same factor.

Various calculations were carried out to find out whether there was any significant expansion in the conduit and capillary tubing joining the syringe pump and the microchannel structure which was found to be negligible and no particular significance was found from expansion of any other portion of the device. These calculations were performed for typical flexible polymer capillaries of which the conduit of the liquid outlet link assembly is made.

The use of a bubble of air is advantageous as heretofore the removal of air has been a major aim of anybody operating in these systems. Using what effectively heretofore was something that you did not require and indeed actively tried to eliminate is advantageous. It would be totally wrong to suggest that the use of the air bubble in accordance with the present invention, is in any way similar to the air bubbles which are sometimes used to separate system and sample liquids within a pumping unit. The amount of air used is substantially greater than would be used in such systems and indeed the air bubble used in the present invention is an air bubble of a precise size to accommodate certain particular situations. The purpose of the air bubble is also different. In the conventional system, it is to be inserted between the system liquid and sample liquid and no other place. In our invention, the air bubble can be inserted in several places and indeed usually not between system liquid and sample liquid. It will be appreciated that other devices for pressure control could be used. A typical example that would be immediately apparent to those skilled in the art, is any form of flexible and elastic membrane. All that is required is to choose the correct material for the membrane and the correct area of it. Also, more sophisticated expansion means and pressure release means could be provided, however, the use of a bubble of air is particularly advantageous.

Calibration of the positive displacement pump 51 and liquid outlet link assembly 50 can be easily carried out by sealing the liquid delivery port 37 and the internal volume of the air bubble can then be determined. By displacing different volumes of liquid from the syringe pump and reading the pressure, one can obtain a calibration curve and then by using the formula (1) above, calculate the volume of air in the system. The internal volume of the bubble includes the volume of air in the liquid reservoir and in the system itself. Such air may be trapped in the pump, tubing, valves, etc. There could be numerous air pockets around different parts of the liquid link assembly which will not cause any difficulty to the operation of the invention in contra distinction to present situations. After calibration, it is possible to adjust the volume of the air in the whole system, thus defining its expandability.

In a typical embodiment, a total volume of 50 μl of liquid was introduced into the syringe pump. The volume of air bubble was between 40 and 120 μl. The typical pressure at the entry of the microstructure was 0.5 to 0.1 mbar, the regulation of flow rate being dependent on the dimensions of the microstructure. For example, for channels of length 20 cm and diameter of 50 μm, the corresponding lowest flow rate that could be achieved was 100 pl/min.

FIG. 14 illustrates theoretical and experimental results for the dependence and the pressure at the entry of the microchannel structure on the volume displaced by the syringe pump. This shows that pressure values can be reasonably well predicted. Thus, once the initial pressure and air bubble volume is known, then it is possible to find required displacement to achieve a desired pressure at the entry port. Again, since

$$pV = constant , and p = \frac{{p}_{0} {V}_{0}}{{V}_{0} - Δ V}$$

where ΔV is the displaced volume, V₀ is initial volume of the bubble under atmospheric pressure, p₀ is the initial pressure, then it is possible, as can be seen, to detect clearly the displacement of the syringe required.

FIG. 15 illustrates this for different volumes of air bubble. It will be seen from this that as the initial volume of the air bubble is increased, this causes decrease in pressure at the entry port of the structure for the same displaced volume of the syringe pump. FIG. 15 shows dearly how one can determine the volume of the bubble and hence the expandability of the system by this calibration.

A positive displacement pump can operate with complex microchannel structures containing several interconnecting channels with complicated geometry. Therefore the simple model for the flow velocity applied to a circular channel is not always valid for such complex structures. In this case, the characteristic parameter of the microchannel structure can be defined as a ratio between flow rate delivered to the microchannel structure and corresponding pressure at the input port We shall call this parameter fluidic resistance R_{f}.
p=R_{f}·Q
where p is a pressure at the input port of the microchannel structure, Q is a flow rate through the microchannel structure.

The concept of fluidic resistance can be essentially true for any Newtonian liquid and any microchannel structure having even complex geometry. fluidic resistance contains information about all geometrical parameters of the microchannel structure.

Once this coefficient is determined, the pressure at the input port can always be calculated given the required flow rate in the microchannel or required linear velocity of the flow in the microchannel. Although the fluidic resistance R_{f} can be calculated analytically from the geometrical parameters of the microchannel structure, it is more practical to determine it experimentally.

Here we describe the method of experimental determination of the fluidic resistance R_{f}. Referring again to FIG. 13, let's assume that strokes of the syringe plunger are periodic and each second ΔV volume of liquid is displaced. The initial volume of air bubble is equal V₀. Each periodic displacement of liquid causes the air bubble to shrink and at the same time, produces the pressure increase at the entry port of the microchannel structure. Due to the increase in pressure, there will be established the liquid flow through the microchannel structure. Its clear that the pressure at the input port will continue to increase until such time, when the flow rate of liquid through the microstructure Q will be equal to the flow rate Q_{plunger} delivered by plunger (volume expelled by the plunger divided by the time of measurements). On the other hand, if the flow rate Q is greater than the O_{plunger}, volume expelled by the plunger divided by the time of measurements, then the pressure at the input port will decrease. Once this stable condition is achieved, the pressure p at the input port can be measured by means of pressure sensor 74. Knowing that Q=Q_{plunger}, we can calculate R_{f}=p/Q=p/Q_{plunger}. The flow rate Q_{plunger} is readily known from the volume of the syringe and the velocity of the plunger that is determined by its controller 5.

Suppose now that we need to establish a particular value of flow rate Q₁ through the microchannel structure. Then, according to the previously measured value R_{f}=p/Q=p/Q_{plunger}, the corresponding pressure p₁ to the flow rate Q₁ at the input port of the microstructure should be:
p₁=R_{f}·Q₁=p·Q₁/Q_{plunger}

In practice, it is convenient to select Q_{plunger} to be in the range of the orders of microlitres/min. This decreases the time required to reach the stable condition of the flow in the microchannel structure. At the same time, Q₁ could be as low as picolitres/min.

It should be noted that the ability to determine the parameters of the microchannel structure during the pump operation allows one to work with microchannels having various geometries and also liquids with different viscosities. This makes the pump independent of the microchannel structure assembly and widens the areas of its application.

The performance of the liquid delivery unit is defined by the software feedback algorithm employed in order to stabilize pressure at the input port of the microchannel structure. It is advantageous to explain in more detail the means of initiation and support of the flow in the microchannel structure. As was shown above, for any given flow rate through the microstructure, the corresponding pressure at the input port can be obtained. Initially, flow is started by displacing the required amount of liquid instantly in such a way that the air bubble compresses and shrinks and produces the required pressure at the input port. Following the initiation, the flow can be supported by the negative pressure feedback, so that when the pressure falls down below required value, the plunger displaces an additional amount of the liquid in order to increase the pressure. On the contrary, when the pressure increase above the required value, the pump reverses and displaces back some volume of liquid and the pressure drops at the input port. Although this simple algorithm allows supporting the flow, oscillations of the flow rate may occur due to the nonlinear response of the pumping system coupled with the expandable element such as air bubble. Backlash of the syringe plunger movement adds additional instability.

Alternatively, in order to facilitate the continuous flow of liquid with predetermined flow rate through the microchannel structure, another approach can be used. The stability of pressure at the input port of the microchannel structure can be achieved by changing the delay between the strokes of the syringe plunger. This alters the flow rate delivered by the plunger Q_{plunger} and accordingly the pressure at the input port. As was mentioned before for the typical operation with a microchannel structure, the strokes of the syringe plunger required to maintain the flow are rather infrequent and therefore delay between them can be set with great precision. The rate of the displacement and pressure can also be used as parameters for PID (proportional-integral-differential) controlled feedback.

Many alternative constructions of liquid delivery unit may be provided and indeed many forms of pumps or more than one pump may be used.

Prior to describing some other embodiments, it is advantageous to discuss some of the tests that may be carried out For example, the manner of operating the embodiment of the previous drawings to study the flow, rolling and migrations of cells, the cell-ligand binding can be achieved in the manner previously described.

In the drawings of FIGS. 16 to 20, the cells are identified by the reference letter C and by suitable lowercase lettering in brackets. Similarly, the arrow F indicates the direction of flow of the liquid sample and the letter L identifies ligand.

Referring to FIG. 16, cells C(a) can be observed as flowing normally through the microchannel 21 while finally the cell C(c) is starting to adhere to the ligand L. Under the right conditions, this observation takes place at some location in each biochip 20 which is being examined.

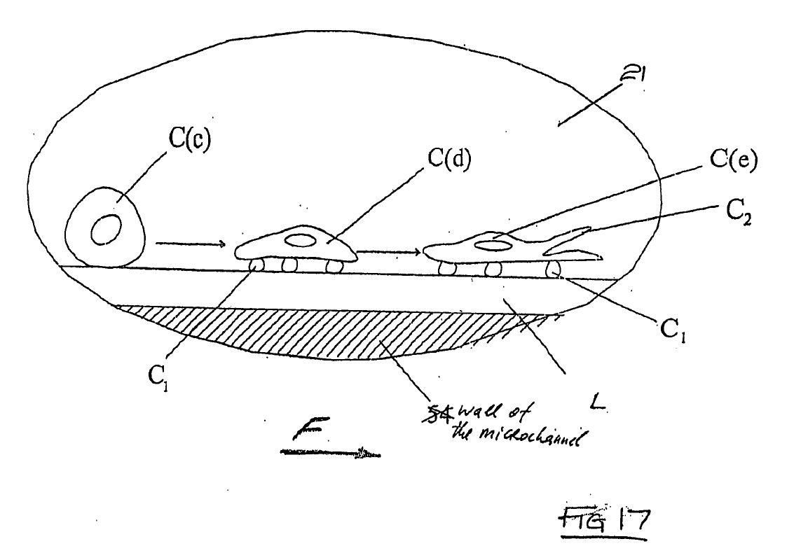

Referring now to FIG. 17, the cell C(c) is shown just beginning to attach to the ligand L. The cell C(d) is shown adhering strongly to the ligand L, in this case, the protein, on the wall of the microchannel 21 with lamellipod/filopod or adhesion plaques, identified by the reference C₁. Finally, the cell C(e) is shown starting to migrate on the ligand L with the leading edge of the cell C(e) starting translocate across the ligand L with a lamellipod/filopod C₁ which elongates and breaks its contact with the ligand L.

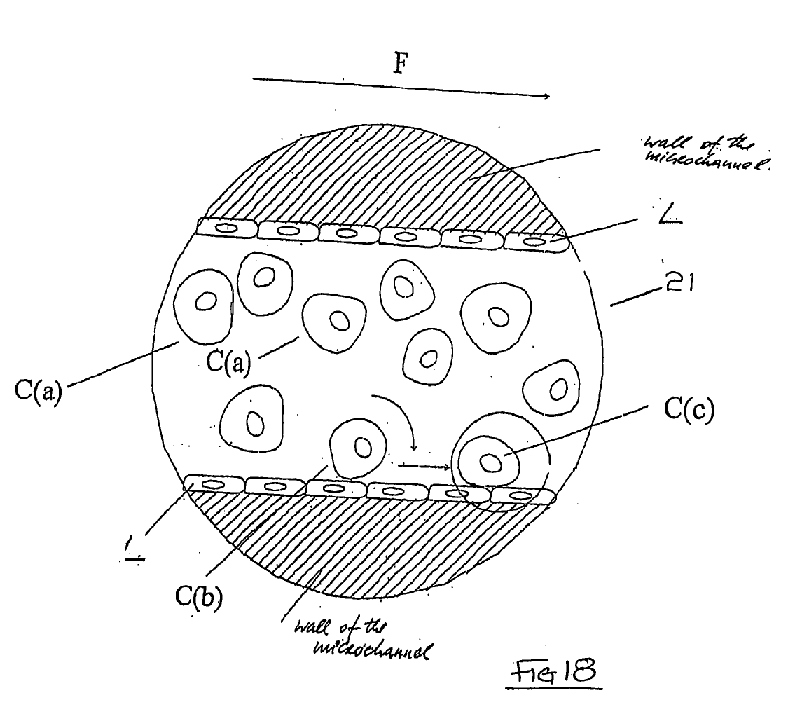

Referring now to FIG. 18, in this assay, the ligand was provided by the seeding and subsequent growth of endothelial cells. This ligand is shown and identified by the letter L and the cells are identified by the same reference numerals. Strictly speaking, the ligands which are available to bind to the receptors on the cells C(c) are on the surface of the endothelium cells. Endothelial cells were chosen as a HUVEC cell line.

Therefore, variations of the test can be carried out such as, for example, assaying one cell type and several ECM ligands. Then each of the biochips 20 would be coated with a different adhesion mediating ligand from the wells 30. Using one liquid delivery unit 3, you inject ligands into each of the inlet ports 22 and, from there, into the biochips 20. Having coated all the microchannels with the chosen ECM ligands, the specified cell type is then injected through each biochip 20. This allows the researcher to build up a profile of the characteristic behaviour of a cell type in response to particular ECM ligands. The same test can then be carried out using different cell types and one ECM ligand. This will allow the option of classifying an ECM ligand according to the behaviour of different cell types with regard to the multistep progress of rolling, tethering, adhesion and subsequent migration. Similarly, this can be done for several cell types with the one endothelial layer.

It is possible to carry out a cell binding assay to identify proteins which will cause specific adherences of particular cell types. From the known initial concentration of cells passed through the biochip during the course of the assay, it is possible to obtain an accurate statistical and qualitative result regarding the percentage of cells which adhered to the coated walls, providing a clear quantitative result for the adhesion affinity of a specific ECM ligand. Here the adhesion affinity refers to the response of cell by adhesion to the ECM ligand-coated channel; i.e. the greater the number of cells adhered to a particular ECM ligand, the greater the adhesion affinity of that ligand. In addition, knowing the velocity of cells within the channels and the length of the channels themselves, it is also possible to obtain a clear physical result regarding the response time of the cell type to its environment. Thus, it is possible to calculate how long it takes the cell to react to its surroundings based on its site of adhesion within the microchannel structure, for example, a cell type that has attached to the chosen ECM ligand or ligands, coating the microchannel walls. Image acquisition and recognition software may be employed to execute an automated based image acquisition or recognition of the cell type or indeed carry out any form of manual cell count.

Thus, for example, it is possible to do any of the following tests:
One cell type and one ECM ligand
One cell type and endothelium layer ligand
One cell type and several ECM ligands
Several cell types and one ECM ligand
Several cell types and endothelium layer ligand

Obviously, various other variations, for example, various cell types and many ligands may also be used. The permutations and combinations are endless.

Finally, the binding affinity can be calculated from the shear stress required to cause dissociation of bound cells. By increasing the flow velocity in the microchannel until there is dissociation of cells from the walls, it is possible to get a measure of the relative binding strengths of various ligands. Therefore, from the strength of the shear stress or corresponding velocity causing dissociation, this can be related to the binding affinity which a particular cell type has for a corresponding adhesion-inducing and mediating ligand. Needless to say, this could be applied to all the assays that have been carried out already. Any flushing liquid may be used, even the sample liquid itself.

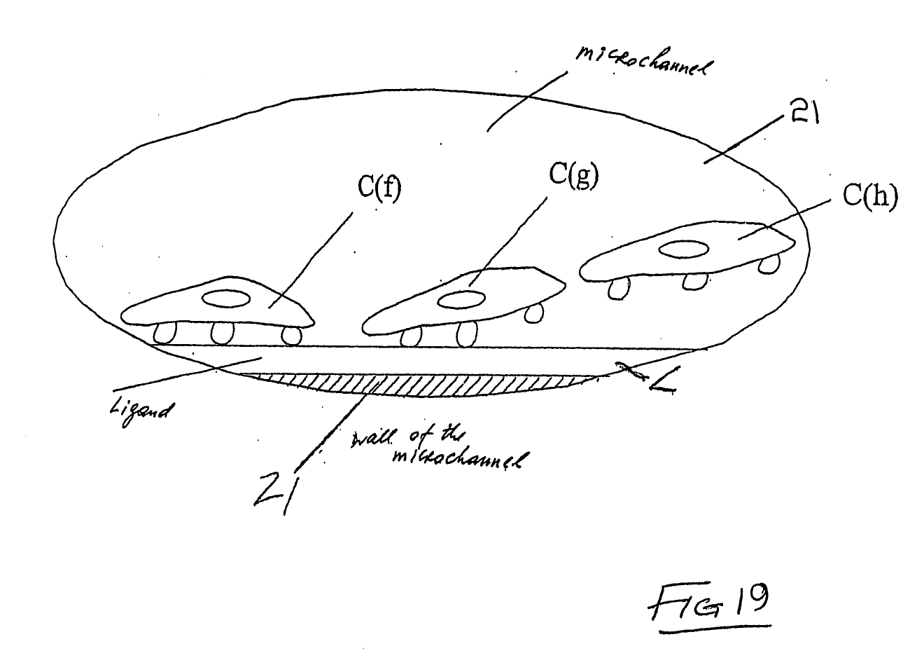

Referring now to FIG. 19, there is illustrated another assay in a view similar to FIG. 16 in which parts similar to those described with reference to the previous drawings are identified by the same reference numerals. In this assay, following adhesion of the cell type to the corresponding adhesion-inducing and mediating ECM ligand, an adhesion-inhibiting reagent, recombinant or cell derived is used. The cell C(f) can be seen securely anchored to the ligand L, then as C(g) beginning to separate and finally at C(h) having separated totally from the ligand. After the dissociation of the cell type from the chosen ECM ligand coating the microchannel walls, it is possible to use image acquisition/recognition software to do an automation based, image acquisition/recognition of cell type or manual cell count to calculate how many cells have responded by clear dissociation from the adhesion-inducing/mediating ECM ligand(s), again providing a clear result for the dissociation affinity of a specific reagent. Here the dissociation affinity refers to the response of a cell by dissociation from the ECM ligand-coated channel; i.e. the greater the number of cells dissociated from the particular ECM ligand, the greater the dissociation affinity of that reagent. Since the percentage of cells from the initial sample of known cell concentration is known, the dissociation affinity results in determination of the percentage of the adhered cells which subsequently dissociated. An identical test can be done for an endothelium layer and one detachment reagent. Then, using the assay assembly 60, many variations on the test can be carried out which will be easily apparent, whether they be one cell type and several ECM ligands and one or more detachment reagents; one ECM ligand, several cell types and one or more detachment reagents; several cell types, one endothelium layer and one or more detachment reagents. Obviously, all these variations will be readily apparent once it is appreciated that the assay assembly is available.

Referring to FIG. 20, there is illustrated an alternative construction of biochip assembly, again identified generally by the reference numeral 20 and parts similar to those described with reference to the previous drawings are identified by the same reference numerals. In this embodiment, each biochip 20 comprises an elongate microchannel, again identified by the reference numeral 21, having intermediate portions 21(a) which have a bore of different cross-sectional area to that of the rest of the microchannel 21. Essentially, the biochip of FIG. 20 is one in which the width of the channel changes from some 200 micrometres to some 50 micrometres. In this particular embodiment, the depth of the microchannel is constant all throughout its length, although channels with varying depth can also be devised. Such a biochip can be particularly useful for applications in assays where the flow under the conditions of the changing shear stress is to be studied. It can mimic the flow of cells in the blood vessel having constrictions.

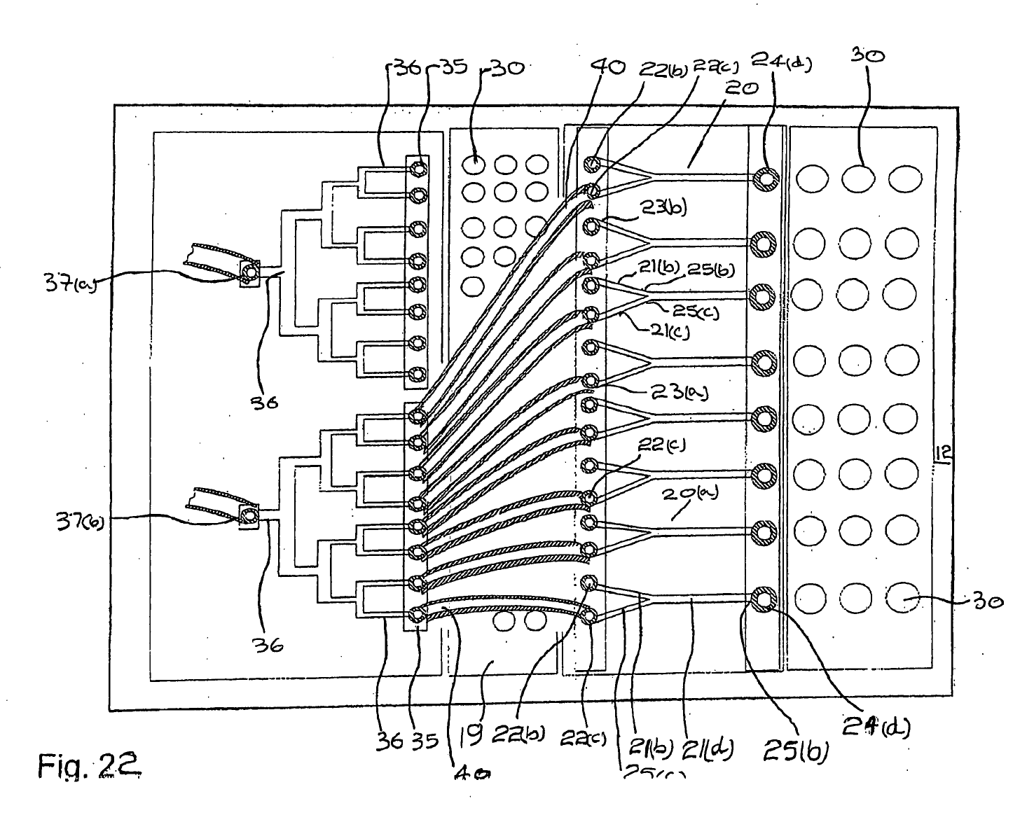

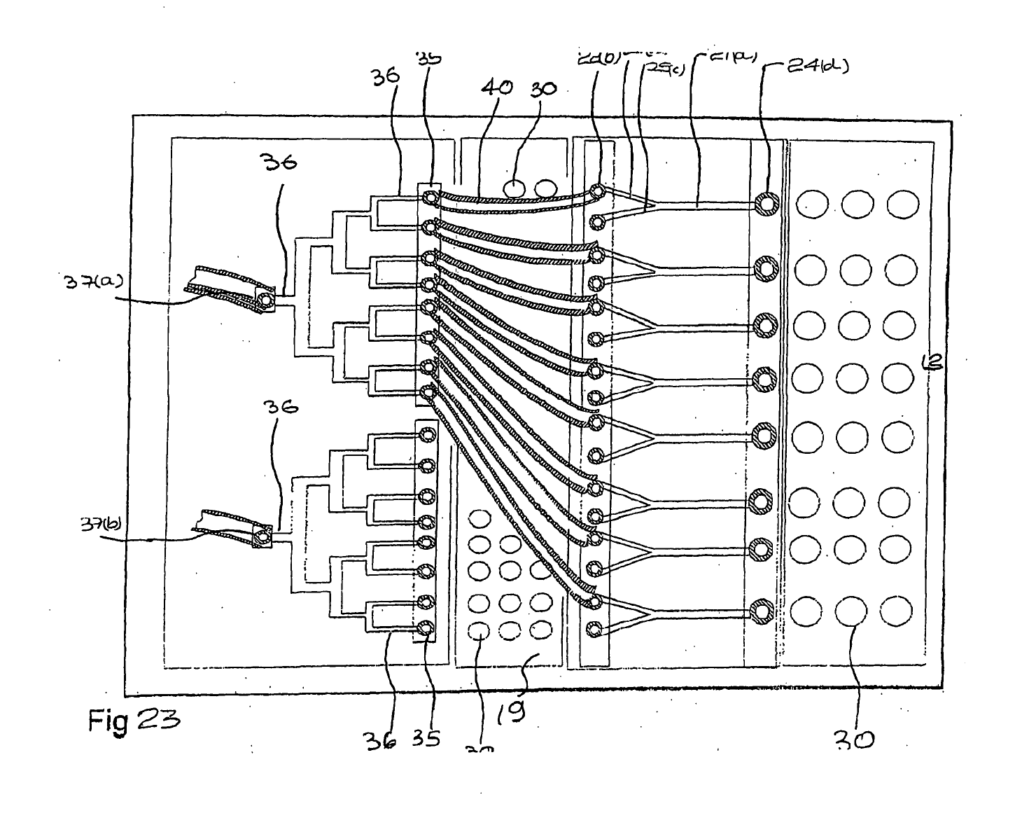

Referring to FIGS. 21 to 23, there is illustrated another assay assembly, identified by the reference numeral 2, in which parts similar to those described with reference to the previous drawings are identified by the same reference numerals. In this embodiment, the biochip 20 comprises a pair of elongate microchannels 21(b) and 21(c), each of which has an inlet 22(b) and 22(c) respectively which, at their distal ends 25(b) and 25(c), are connected together into a further microchannel 21(d) having an outlet port 24(d) at its distal end 25(d) to form therewith a Y-shaped composite microchannel 21(b), (c) and (d). There are two liquid delivery ports 37(a) and 37(b) which are fed from two separate liquid delivery units (not shown). FIGS. 22 and 23 show how, with the use of the transfer conduits 40, the various inlet ports 22(b) and 22(c) can be fed individually.

This will allow other assays to be carried out. Conduits connected to outlet ports 24(d) are not shown here for simplicity.

FIG. 24 illustrates the layout of a biochip assembly, again identified by the reference numeral 2. Again, parts similar to those described with reference to the previous drawings are identified by the same reference numerals. This simply illustrates that the individual biochips 20 do not have to be arranged in line.

Referring to FIGS. 25 to 27, there is illustrated a fully assembled biochip assembly, in one position of use and prior to the fitting of most of the transfer conduits 40, again indicated generally by the reference numeral 2. There is now illustrated the upper support plate 46 mounted in spaced-apart relationship with the biochip sheet 15. As described above, the upper support plate 46 has a plurality of rigid tubes 45 mounted in it. The tubes 45 project proud of the upper face 47 and the lower face 48. Each tube 45 projects proud of the upper face 47 for connection to one of the transfer conduits 40 and at its other end below the lower face 48 for connection to one of the ports 22, 24 and wells 30, FIG. 26 showing it about to be connected to an inlet port 22.

Releasable connection means, indicated generally by the reference numeral 63, is provided for mounting the plate 46 above the top face 12 of the biochip sheet 15. The releasable connection means 63 comprises a pair of spaced-apart support columns 65 projecting up from and thus proud of the biochip sheet 15 and mounting a pivot bar 66 therebetween. A support member 67 is pivotally mounted on the bar 66 and houses an open-mouthed slot 68 for reception of the plate 46. Portion of the support member 67 forms a camming surface 69 for engaging the top face 12 of the biochip sheet 15.

It will be appreciated that the pivoting of the support member 67 in the direction of the arrows A will cause the camming surface 69 to bear against the upper face 12 of the biochip sheet 15 and thus secure the plate 46 in position. Further connection can then be made.

Referring to FIG. 27, there is illustrated an alternative construction of biochip, again identified by the reference numeral 20, in which there is also included an inlet gas venting port 22(f) and an outlet gas venting port 23(f).

FIG. 28 shows another construction of biochip, again indicated generally by the reference numeral 20, which comprises a pair of elongate microchannels 21(g) and 21(h) joined together by a further microchannel 21(j) intermediate their proximal and distal ends.

Various methods may be provided for cleaning the transfer conduits and ensuring that there is no cross contamination between samples. For example, the conduit can simply be removed and disposed of after it has been used to aspirate and deliver any one liquid, whether it be a reaction liquid, a cell based liquid or a ligand. Then, the transfer conduit can be easily cleaned. Alternatively, system liquid can be used as a flushing liquid. Many other ways may be provided.

In the embodiments described above, there is described the use of a tightly controlled volume of air bubble to provide pressure stabilising means. It will be appreciated that this can be many bubbles and they do not all have to be mounted, for example, in the one particular part of the link body. Further, it is envisaged that the link body itself could provide the pressure compressible means by simply having a link body of a suitable material which would expand and contract, depending on the pressure exerted. Further, various forms of membrane could also be used.

It will be appreciated that with some of the embodiments now described, various other assays may be carried out.

It will be appreciated that sample liquids from the assays may be collected in the wells at the end of the assay for any post-assay analysis tests that may be required. For example, this could be mass spectrometry analysis, chromatography or another chemical or biochemical analysis.

It will also be appreciated that although the biochip assemblies above show eight biochips therein, other numbers of biochips could be provided.

The biochips are fabricated using standard lithographic and hot embossing techniques. A stainless steel substrate is masked with photoresist (SU-8-5μm, Chestech). After ultraviolet lithography, the photoresist mask is developed and the substrate is electrochemically etched to produce a negative master mould in stainless steel; The remaining mask is subsequently removed. Hot embossing is employed to replicate the microfluidic pattern of the microchannels in a variety of thermoplastic materials such as PMMA, polycarbonate, and polystyrene. The fluidic connection ports, comprising eight connections in parallel are glued in position at the exit of the flow splitter, i.e. the main feeder channels 36, and at the input and output of the analysis section. A single connection port is glued at the input of the flow splitter to provide the liquid inlet port 37. Microwells for the preparation of the sample and collection after the analysis of said sample are introduced via similar hot embossing procedures using a specifically designed microwell-mould.

The biochip is treated in oxygen plasma (0.1 torr, 80% oxygen and +100V for 30 seconds) to ensure a hydrophilic surface and is subsequently sealed with a pressure-sensitive film (ARCLEAR 8796, Adhesives Research Inc.). This film is a 3.0-mil (75 μ) optical grade polyester film coated on one side with an optically clear pressure sensitive adhesive. It has a high bond level to many different surfaces, offering virtually defect-free bonding to flexible or rigid optical components. The film can be removed after the execution of an assay and thus it is possible to inject a solution that fixes cells to the film and the plastic substrate of the biochip enabling further study. The film may be removed and the cells taken away for additional research.

The width of the channels may vary in the range of 5 to 500 μm and a depth, in the range of 15 to 50 μm but generally the cross-section will exceed 20 μm×20 μm. The biochip is thus an optically transparent structure. They can be of any shape, such as straight sided, arcuate or cylindrical in cross-section.

It will be appreciated that to a certain extent, the term “input port” and “output port” is a misnomer since in one circumstance, a port may operate as an input port and in another circumstance, as an output port.

It is well known that there are some essential nutritional requirements for living human cells and standard culture medium was used. A minimal medium contained glucose as a source of carbon, NH₄Cl as the source of nitrogen and salts such as Na⁺, K⁺, Mg⁺, Ca⁺, SO₄²⁻, Cl and PO₄³⁻. In certain circumstances, in carrying out the tests, when a richer culture medium was required, partly hydrolysed animal or plant tissues rich in amino acids, short peptides and lipids, were used, as well as yeast extract which is rich in vitamins and enzyme cofactors, nucleic acid precursors and amino acids.

One of the major difficulties in carrying out an assay in the biochip format is to ensure that the flow rate was kept as constant as possible. The problem with variations in flow rates is that they can provide variations in the shear stress on the wall, for example, of a capillary or of a microchannel such as in accordance with the present invention. Typical flow rates in the cell based assays are in the range from 100 pl/min to 10 l/min. The corresponding linear velocities for these flow rates were 0.5 μm/s to 5 cm/s respectively. This is achieved by using the liquid delivery unit according to the invention.

One of the great advantages of the cell based assay assembly that will become apparent is that a variety of tests can be carried out. However, there is a further advantage in that since these tests occur over relatively long periods of time, of the order of hours or so, it is possible to use the one microscope to carry out a multiplicity of examinations as it is usually only necessary to have the activities recorded at discrete time intervals. Thus, for example, the microscope 7 of FIG. 1 can be indexed to examine each of the biochips 20 by simple manipulation. For this, the biochip assembly 2 is positioned on an XY table that moves the biochip assembly 2 with respect to the objective of the microscope 7 so that any locations of any microchannel 21 can be inserted in the focus of the microscope 7. Further, it will be appreciated that assemblies with greater than eight separate biochips mounted thereon, may be advantageous. By using relatively large biochip assemblies, that is to say, containing a multiplicity of individual biochips and using the one microscope, it should be possible to carry out assays with greater number of samples.

In the assays described, the microchannels were comparable in size to the post capillary venules in the human bodies and therefore it is suggested that the microchannels imitate the natural environment more closely than any other form of channel. Thus, when dealing with assays concerning venules in the human body, sizes are of the order of 20 μm while for human capillaries, they can be as small as 8 μm.

In the embodiments described, a pressure-sensitive adhesive coated film is used to cover the biochips 20 effectively sealing the microchannels. Thus, the pressure-sensitive film can be removed after the execution of an assay and accordingly it is possible, prior to removal of the film, to inject a solution which fixes cells to the film and the plastic substrate of the biochip enabling further study. The pressure-sensitive adhesive coated film may be removed and the cells adhered to it taken away for additional research.

Needless to say, in the embodiments described above, the length of the microchannel has been greatly foreshortened, however, it will be appreciated that the microchannels can be lengthened by intertwining microchannels within each other or making them, for example, in the configuration as shown in FIG. 26. Essentially, the microchannel can be folded In on itself so that a longer microchannel can be accommodated on the one sheet with the same footprint. All the various constructions of microchannel are not illustrated as they will be readily easily appreciated by those skilled in the art and particularly by those who wish to manufacture such microchip assemblies.

It will be readily appreciated that cellular activation can also be studied using the present invention. The purpose of such an assay is to determined if the nature of the cell (e.g. lymphocyte) activation determines binding specificity or preference for either the ECM ligand or an individual chemoattractant migratory signals. In this case, the microchannels of each individual biochip 20 are individually coated with specific matrix ligands, e.g. fibronectin, collagen or hyalaronic acid. Depending on the nature of the microchannel, the cells can be permitted to crawl through a protein coated channel before encountering multiple channels coated with individual matrix molecules by using different constructions of biochip, as will be appreciated from the various embodiments described.

It will be appreciated that it would be possible to use a plurality of biochips in series. Thus, for example, rather than one array of biochips in parallel, as illustrated, there could be further arrays of biochips to form the biochip assembly.

It will be appreciated that the transfer conduits are essentially disposable sample holders. It will also be appreciated that in most cases, biological assays are a multi-stage process and thus requires consecutive injection of several samples into the one microchannel. Thus, an ability to dispose of the sample holder tube or conduit contaminated with one sample and replace it with a new uncontaminated tube, is particularly important. It is also important to avoid the contamination of any of the other parts of the biochip and thus cross contamination.

It will be appreciated that the biochips incorporated can be any of the biochips as previously described.

It will also be appreciated that it is advantageous to be able to collect the samples from the output of the analysis section, that is, where the biochips 20 are situated. In many situations, for example, gene expression of sample cells which did not react with a particular ligand may be required. Similarly, waste ligand solution can be stored in one of the output reservoir wells. It will also be appreciated that additional reservoir wells may be provided and that further, additional sets of biochips may also be provided.

One of the great advantages of using the biochip assembly in accordance with the present invention is the reduction in reagent or sample consumption. It will also allow reduced analysis times and larger transfer rates due to the diminished distances involved. Additionally, in running several assays in parallel, each process in an assay can be manipulated step by step through computer control enabling great efficiency. Again, this accuracy in combination with higher yields, leads to a reduction in waste. This is not only more economically favourable but also environmentally beneficial where hazardous chemicals are involved.

In addition to chemical production, there are numerous other fields in which the micro devices according to the present invention can make a contribution, such as microbiology, pharmacy, medicine, biotechnology and environmental and materials science. The present invention is particularly adapted to the field of drug discovery and combinatorial chemistry. Again, there should be considerable cost savings for pharmaceutical companies. One of the great advantages of the present invention is that it mimics in vivo testing. Obviously, with the present invention, there is a constant flow of cells and the drug candidate, together with the micro capillary under observation, produces much more accurate statistical results.

One of the problems with current toxicity tests is that the systems implemented are not always representative of those in vivo providing results which are not characteristic of the in vivo situation. Secondly, there are differences with culturing and maintaining certain cells in vitro. The present invention allows one to simulate in vivo conditions eliminating many of the disadvantages of the present testing and hence immediately decreasing the necessity for animal trials while simultaneously increasing the statistical response as a result of the continuous flow assay according to the present invention.

One of the major problems with all drug testing is that clinical trials involve testing of the new drug in humans and because of the rigorous testing involved in a new drug, the time and cost of bringing a drug to market is enormous. It is for this reason that pharmaceutical companies must be extremely accurate with results obtained through experimental assays before presenting a new drug for clinical trials.

One of the advantages of the present invention is that relatively small volumes of blood can be used for analysis in hospitals which can be extremely advantageous. A particular advantage of the present invention is that the biochips are disposable.

The present invention essentially provides techniques for performing assays that test the interaction of a large number of chosen compounds, for example, candidate drugs or suspected toxic samples with living cells while the cells and/or the compounds mimic the in vivo situation of continuous flow. The assays according to the present invention imitate as far as possible the natural situation, while additionally overcoming the disadvantages of other techniques resulting in a fast and accurate process.

It will be appreciated that since the biochips are fabricated from a plastics material, it is considerably less expensive than, for example, silicone micro-machining which is often used at present, for such microchips.

One of the great advantages of plastics material is that it enables real-time monitoring with relative ease, by use of a inverted microscope.

The size of the microchannels is also significant Dimensions below the order of 1 mm have long be avoided due to the many difficulties that occurred when scaling down. Such difficulties involve the control of flow within these microchannels.

While in the present invention, many tests have been tried and described, it will be appreciated that many other assays and tests can be carried out in accordance with the present invention. Indeed, some of the tests according to the present invention are not so much tests, as indeed filtering operations.

In the specification the terms “comprise, comprises, comprised and comprising” or any variation thereof and the terms “include, includes, included and including” or any variation thereof are considered to be totally interchangeable and they should all be afforded the widest possible interpretation.

The invention is not limited to the embodiments hereinbefore described but may be varied in both construction and detail.

1-46. (canceled)

47. A biochip assembly for a cell based assay comprising:
a plurality of biochips, each biochip comprising at least one elongate microchannel,
an inlet port adjacent one end of each microchannel,
an outlet port adjacent the other end of each microchannel;
a liquid delivery unit having a plurality of liquid delivery ports, one for connection to each biochip;
at least one fluidly separate reservoir well for use with each biochip; and
releasable connection means for each port and well for reception of removable separate enclosed transfer conduits for releasable fluidic connection of some of the ports to other of the ports and wells.

48. A biochip assembly for a cell based assay comprising:
a plurality of biochips, each biochip comprising at least one elongate microchannel,
an inlet port adjacent one end of each microchannel and an outlet port adjacent the other end of each microchannel;
a liquid delivery unit having at least one liquid delivery port for connection to each biochip;
two sets of at least two fluidly separate reservoir wells, one adjacent the inlet port and the other adjacent the outlet port of each biochip; and
releasable connection means for each port and well for reception of removable separate enclosed transfer conduits for releasable fluidic connection of some of the ports to other of the ports and wells.

49. A biochip assembly for a cell based assay comprising:
a plurality of biochips, each biochip comprising at least one elongate microchannel, an inlet port adjacent one end of each microchannel and an outlet port adjacent the other end of each microchannel;
a liquid delivery unit having a plurality of liquid delivery ports, one for connection to each biochip,
at least one fluidly separate reservoir well for use with each biochip; and
a plurality of removable separate enclosed transfer conduits for releasable fluidic connection of some of the ports to other of the ports and wells.

50. The biochip assembly as claimed in claim 49, in which each biochip has more than one inlet port, each of which is for connection to a different liquid delivery unit.

51. The biochip assembly as claimed in claim 49, in which each biochip has more than one outlet port.

52. The biochip assembly as claimed in claim 49, in which the biochip comprises a pair of elongate microchannels, each having at least one inlet port at its proximal end and at their distal ends connecting into a further microchannel having at least one outlet port at its distal end to form therewith a Y-shaped composite microchannel.

53. The biochip assembly as claimed in claim 49, in which the biochip comprises at least one elongate microchannel having a bore, at least one intermediate portion of which has a different cross-sectional area to that of the rest of the microchannel.

54. The biochip assembly as claimed in claim 49, in which each biochip comprises a pair of elongate microchannels, each microchannel having at least one inlet port and at least one outlet port, the microchannels being connected their proximal ends and distal ends.

55. A biochip assembly for a cell based assay comprising:
a plurality of biochips, each biochip comprising at least one elongate microchannel, an inlet port adjacent one end of each microchannel and an outlet port adjacent the other end of each microchannel;
a liquid delivery unit having a liquid delivery port for connection to each biochip;
at least one fluidly separate reservoir well for use with each biochip; and
a plurality of removable separate enclosed transfer conduits for releasable fluidic connection of some of the ports to other of the ports and wells, each transfer conduit having an internal cross sectional area substantially greater than that of each microchannel.

56. A biochip assembly for a cell based assay comprising:
a plurality of biochips, each biochip comprising an elongate microchannel, an inlet port adjacent one end of each microchannel and an outlet port adjacent the other end of each microchannel;
a liquid delivery unit having at least one liquid delivery port;
two sets of at least two fluidly separate reservoir wells, one adjacent the inlet port and the other adjacent the outlet port of each biochip; and
a plurality of removable separate enclosed transfer conduits for releasable fluidic connection of some of the ports to other of the ports and wells, each transfer conduit having an internal cross sectional area substantially greater than that of each microchannel.

57. A biochip assembly for a cell based assay comprising:
a planar biochip sheet of translucent material or combination of several materials;
a plurality of biochips, each biochip comprising at least one elongate microchannel formed by open cut-out channels in one bottom face of the biochip sheet covered by a thin film of material or several materials one on top of another;
an inlet port for each microchannel in the top face of the biochip sheet communicating adjacent one end of the microchannel;
an outlet port for each microchannel in the top face of the biochip sheet communicating adjacent the other end of the microchannel;
a fluidly separate reservoir in the top face adjacent each inlet and outlet port;
a liquid delivery unit having a liquid delivery port for connection to each biochip; and
releasable connection means for each port and well for reception of removable separate enclosed transfer conduits for releasable fluidic connection of some of the ports to other of the ports and wells.

58. The biochip assembly as claimed in claim 57, in which the microchannels are of non-cylindrical cross-section.

59. A biochip assembly for a cell based assay comprising:
a planar biochip sheet of translucent material or combination of several materials;
a plurality of biochips, each biochip comprising at least one elongate microchannel formed by open cut-out channels in one bottom face of the biochip sheet covered by a thin film of material or combination of several materials;
an inlet port for each microchannel in the top face of the biochip sheet communicating adjacent one end of the microchannel;
an outlet port for each microchannel in the top face of the biochip sheet communicating adjacent the other end of the microchannel;
a fluidly separate reservoir in the top face adjacent each inlet and outlet port;
a liquid delivery unit having a liquid delivery port;
a main liquid feeder channel comprising an open cut-out liquid channel in the bottom face for connection to the liquid delivery port of the liquid delivery unit and having a plurality of delivery ports in the top face, one for each microchannel and for the feeder channel, the liquid channel being covered by a thin film of material or combination of materials; and
releasable connection means for each port and well for reception of removable separate enclosed transfer conduits for releasable fluidic connection of some of the ports to other of the ports and wells.

60. A biochip assembly for a cell based assay comprising:
a planar biochip sheet of translucent material or combination of several materials;
a plurality of biochips, each biochip comprising at least one elongate microchannel formed by open cut-out channels in one bottom face of the biochip sheet covered by a thin film of material or combination of several materials;
an inlet port for each microchannel in the top face of the biochip sheet communicating adjacent one end of the microchannel;
an outlet port for each microchannel in the top face of the biochip sheet communicating adjacent the other end of the microchannel;
a fluidly separate reservoir in the top face adjacent each inlet and outlet port;
a liquid delivery unit having a liquid delivery port;
an upper support plate having an upper face and a lower face in use;
a plurality of tubes mounted in the plate and projecting proud of the faces, each tube proud of the upper face being for connection to one of the transfer conduits and at its other end for connection to one of the ports and wells; and
releasable connection means for each port and well for reception of removable separate enclosed transfer conduits for releasable fluidic connection of some of the ports to other of the ports and wells.

61. The biochip assembly as claimed in claim 60, in which releasable connection means is provided for mounting the plate above the top face of the biochip sheet in correspondence with ports or wells.

62. A biochip assembly for a cell based assay comprising:
a planar biochip sheet of translucent material or combination of several materials;
a plurality of biochips, each biochip comprising an elongate microchannel formed by open cut-out channels in one bottom face of the biochip sheet covered by a thin film of material or combination of several materials;
an inlet port for each microchannel in the other top face of the biochip sheet communicating adjacent one end of the microchannel;
an outlet port for each microchannel in the top face of the biochip sheet communicating adjacent the other end of the microchannel;
a fluidly separate reservoir in the top face adjacent each inlet and outlet port;
a liquid delivery unit having a liquid delivery port for connection to each biochip;
a main liquid feeder channel comprising an open cut-out liquid channel in the bottom face for connection to the liquid delivery port of the liquid delivery unit and having a plurality of delivery ports in the top face, one for each microchannel and for the feeder channel, the liquid channel being covered by a thin film of material or several materials;
an upper support plate having an upper face and a lower face in use;
a plurality of tubes mounted in the plate and projecting proud of the faces, each tube proud of the upper face being for connection to one of the transfer conduits and at its other end for connection to one of the ports and wells;
releasable connection means for each port and well for reception of removable separate enclosed transfer conduits for releasable fluidic connection of some of the ports to other of the ports and wells.

63. The biochip assembly as claimed in claim 62, in which the releasable connection means comprises:
a pair of spaced-apart columns proud of the biochip sheet and mounting a pivot bar therebetween; and
a support member pivotally mounted on the bar and having a channel-shaped elongate open mounted slot for reception of the plate, portion of the support member forming a camming surface for engaging the top face of the biochip sheet when pivoted into a position to engage the plate above the biochip sheet.

64. The biochip assembly as claimed in claim 62, in which when the biochips each have additional inlet ports and there are additional sets of main liquid feeder channels, the number of such sets equals the number of additional inlet portions for each biochip.

65. The biochip assembly as claimed in claim 62, in which the inlet ports and outlet ports on the top face have bores between entrance and exit, of substantially constant cross-sectional area and of substantially the same order of magnitude as that of the microchannels.

66. A biochip assembly for a cell based assay comprising:
a plurality of biochips, each biochip comprising at least one elongate microchannel, an inlet port adjacent one end of each microchannel and an outlet port adjacent the other end of each microchannel;
a liquid delivery unit having a liquid delivery port for connection to each biochip, the liquid delivery unit further comprising a liquid outlet link assembly to provide a steady liquid delivery output rate below 10 μl per minute through the liquid delivery port of the liquid delivery unit from a link input port connected to a positive displacement pump forming part of the liquid delivery unit and having an immediate step pumping rate substantially greater than the desired steady liquid delivery output rate, the liquid outlet link assembly further comprising a hollow link body having a resistance to flow therethrough substantially less than through the liquid delivery port and pressure stabilizing means for the link body formed by pressure compressible means connected thereto whereby, on increased pressure being encountered in the hollow link body on operation of the positive displacement pump, the pressure compressible means initially contracts to counteract the pressure rise in the liquid outlet link assembly and hence the rise in the liquid flow rate through the liquid delivery port and then as delivery of liquid takes place through the liquid delivery port the pressure compressible means expands to maintain the pressure within the liquid link assembly relatively stable;
at least one fluidly separate reservoir well for use with each biochip; and
a plurality of removable separate enclosed transfer conduits for releasable fluidic connection of some of the ports to other of the ports and wells.

67. The biochip assembly as claimed in claim 66, in which the pressure compressible means comprises a gas bubble.

68. The biochip assembly as claimed in claim 66, in which the compressible means comprises more than one gas bubble and the aggregate volume of the bubbles is a multiple of the volume of liquid dispensed in one step of the pump.

69. The biochip assembly as claimed in claim 66, in which the compressible means comprises an elastic membrane forming part of the link body.

70. The biochip assembly as claimed in claim 66, in which the link body comprises expandable tubing which forms the expansion means.

71. The biochip assembly as claimed in claim 66, in which control means is provided and is connected to a flow conditions sensing means for the liquid outlet link assembly for causing the pump to operate to provide the desired flow rate through the outlet port.

72. The cell based assay assembly comprising a biochip assembly as claimed in claim 66 and detection and recording equipment for conducting an assay on a biological cell as it is delivered through the biochip assembly.

73. The cell based assay assembly as claimed in claim 72, in which the detection and recording equipment comprises an optically inverted microscope, a digital camera and computerized recording, monitoring and control means.

74. The cell based assay assembly as claimed in claim 72, in which the detection and recording equipment comprises an epifluorescence device.

75. A biochip assembly for a cell based assay comprising:
a plurality of biochips, each biochip comprising at least one elongate microchannel, an inlet port adjacent one end of each microchannel and an outlet port adjacent the other end of each microchannel;
a liquid delivery unit having a liquid delivery port for connection to each biochip, the liquid delivery unit further comprising a liquid outlet link assembly to provide a steady liquid delivery output rate below 10 μl per minute through the liquid delivery port of the liquid delivery unit from a link input port connected to a positive displacement pump forming part of the liquid delivery unit and having an immediate step pumping rate substantially greater than the desired steady liquid delivery output rate, the liquid outlet link assembly further comprising a hollow link body having a resistance to flow therethrough substantially less than through the liquid delivery port and pressure stabilizing means for the link body formed by pressure compressible means connected thereto whereby, on increased pressure being encountered in the hollow link body on operation of the positive displacement pump, the pressure compressible means initially contracts to counteract the pressure rise in the liquid outlet link assembly and hence the rise in the liquid flow rate through the liquid delivery port and then as delivery of liquid takes place through the liquid delivery port the pressure compressible means expands to maintain the pressure within the liquid link assembly relatively constant;
at least one fluidly separate reservoir well for use with each biochip; and
a plurality of removable separate enclosed transfer conduits for releasable fluidic connection of some of the ports to other of the ports and wells, each transfer conduit having an internal cross sectional area substantially greater than that of each microchannel.

76. A biochip assembly for a cell based assay comprising:
a plurality of biochips, each biochip comprising an elongate microchannel;
an inlet port adjacent one end of each microchannel;
an outlet port adjacent the other end of each microchannel;
a liquid delivery unit having a liquid delivery port for connection to each biochip, the liquid delivery unit further comprising a liquid outlet link assembly to provide a steady liquid delivery output rate below 10 μl per minute through the liquid delivery port of the liquid delivery unit from a link input port connected to a positive displacement pump forming part of the liquid delivery unit and having an immediate step pumping rate substantially greater than the desired steady liquid delivery output rate, the liquid outlet link assembly further comprising a hollow link body having a resistance to flow therethrough substantially less than through the liquid delivery port and pressure stabilizing means for the link body formed by pressure compressible means connected thereto whereby, on increased pressure being encountered in the hollow link body on operation of the positive displacement pump, the pressure compressible means initially contracts to counteract the pressure rise in the liquid outlet link assembly and hence the rise in the liquid flow rate through the liquid delivery port and then as delivery of liquid takes place through the liquid delivery port the pressure compressible means expands to maintain the pressure within the liquid link assembly relatively constant;
at least one fluidly separate reservoir well for use with each biochip; and
releasable connection means for each port and well for reception of removable separate enclosed transfer conduits for releasable fluidic connection of some of the ports to other of the ports and wells.

77. A biochip assembly for a cell based assay comprising:
a plurality of biochips, each biochip comprising an elongate microchannel, an inlet port adjacent one end of each microchannel and an outlet port adjacent the other end of each microchannel;
a liquid delivery unit having at least one liquid delivery port, the liquid delivery unit further comprising a liquid outlet link assembly to provide a steady liquid delivery output rate below 10 μl per minute through the liquid delivery port of the liquid delivery unit from a link input port connected to a positive displacement pump forming part of the liquid delivery unit and having an immediate step pumping rate substantially greater than the desired steady liquid delivery output rate, the liquid outlet link assembly further comprising a hollow link body having a resistance to flow therethrough substantially less than through the liquid delivery port and pressure stabilizing means for the link body formed by pressure compressible means connected thereto whereby, on increased pressure being encountered in the hollow link body on operation of the positive displacement pump, the pressure compressible means initially contracts to counteract the pressure rise in the liquid outlet link assembly and hence the rise in the liquid flow rate through the liquid delivery port and then as delivery of liquid takes place through the liquid delivery port the pressure compressible means expands to maintain the pressure within the liquid link assembly relatively constant;
at least one fluidly separate reservoir well for use with each biochip; and
a plurality of removable separate enclosed transfer conduits for releasable fluidic connection of some of the ports to other of the ports and wells.

78. A biochip assembly for a cell based assay comprising:
a plurality of biochips, each biochip comprising an elongate microchannel, an inlet port adjacent one end of each microchannel and an outlet port adjacent the other end of each microchannel;
a liquid delivery unit having at least one liquid delivery port, the liquid delivery unit further comprising a liquid outlet link assembly to provide a steady liquid delivery output rate below 10 μl per minute through the liquid delivery port of the liquid delivery unit from a link input port connected to a positive displacement pump forming part of the liquid delivery unit and having an immediate step pumping rate substantially greater than the desired steady liquid delivery output rate, the liquid outlet link assembly further comprising a hollow link body having a resistance to flow therethrough substantially less than through the liquid delivery port and pressure stabilizing means for the link body formed by pressure compressible means connected thereto whereby, on increased pressure being encountered in the hollow link body on operation of the positive displacement pump, the pressure compressible means initially contracts to counteract the pressure rise in the liquid outlet link assembly and hence the rise in the liquid flow rate through the liquid delivery port and then as delivery of liquid takes place through the liquid delivery port the pressure compressible means expands to maintain the pressure within the liquid link assembly relatively constant;
at least one fluidly separate reservoir well for use with each biochip; and
a plurality of removable separate enclosed transfer conduits for releasable fluidic connection of some of the ports to other of the ports and wells, each transfer conduit having an internal cross sectional area substantially greater than that of each microchannel.

79. The method of conducting a biological cell assay on a cell based assay assembly as claimed in claim 49, comprising the steps of:
(a) connecting the liquid delivery outlet port to a well by a transfer conduit;
(b) aspirating liquid from the well into the transfer conduit;
(c) connecting the transfer conduit to an inlet port;
(d) delivering liquid from the transfer conduit through the biochip and then repeating steps (a) to (d) as often as required; and
(e) then carrying out the assay with the detection and recording equipment as the final step (d) is being carried out.

80. The method as claimed in claim 79, in which the additional step, after one or more of step (d), is carried out of simultaneously using another transfer conduit to connect the outlet port of the biochip to another well.

81. The method as claimed in claim 79, in which the additional step is performed of filling the transfer conduit with the system liquid.

82. The method as claimed in claim 79, in which the additional step is performed of replacing the transfer conduit between aspirating liquids from wells during steps (a)-(d) in order to avoid cross-contamination.

83. The method as claimed in claim 79, in which, after aspirating liquid from a well, the additional step of flushing system liquid through the transfer conduit is carried out.

84. The method as claimed in claim 79, in which a desired flow rate (Q₁) within the biochip assembly is achieved by:
determining the required pressure (P₁) within the liquid delivery unit to achieve the desired flow rate (Q₁) by first determining a steady flow rate (Q_{plunger}) for the pump which maintains a constant pressure (P) within the biochip assembly to provide a fluidic resistance factor (R_{f}) for each biochip determined by dividing the pressure (P) by the flow rate (Q_{plunger}) and then multiplying the desired flow rate (Q₁) by this fluidic resistance factor (R_{f}) to provide the required pressure (P₁); and
then operating the pump to provide the required pressure (P₁).

85. The method as claimed in claim 79, in which a desired flow rate (Q₁) within the biochip assembly is achieved by:
determining the required pressure (P₁) within the liquid delivery unit to achieve the desired flow rate (Q₁) by first determining a constant pressure (P) which maintains a steady flow rate (Q_{plunger}) for the pump within the biochip assembly to provide a fluidic resistance factor (R_{f}) for each biochip determined by dividing the pressure (P) by the flow rate (Q_{plunger}) and then multiplying the desired flow rate (Q₁) by this fluidic resistance factor (R_{f}) to provide the required pressure (P₁); and
then operating the pump to provide the required pressure (P₁).

86. The method as claimed in claim 84 or 85, in which when the pressure drops below the required pressure (P₁) by a predetermined amount, the pump is operated to deliver liquid into the liquid delivery unit and when the required pressure is exceeded by a predetermined amount, the pump is reversed to aspirate liquid.

87. The method as claimed in claim 84 or 85, in which the flow rate of the pump is varied to maintain the pressure within a predetermined range of pressure.

88. The method as claimed in claim 84 or 85, in which the required pressure (P₁) is achieved with the predetermined displacement volume (ΔV) of the pump over a predetermined time by varying the compressibility of the pressure compressible means.

89. The method as claimed in claim 88, in which the varying of the compressibility of the pressure compressible means comprises adding or reducing the amount of gas within the link body.
